(19) United States
(12) Patent Application Publication
(10) Pub. No.: US 2017/0044624 A1
(43) Pub. Date: Feb. 16, 2017

(54) METHODS AND MATERIALS FOR IDENTIFYING AND TREATING MAMMALS HAVING HER2-POSITIVE BREAST CANCER

(71) Applicant: Mayo Foundation for Medical Education and Research, Rochester, MN (US)

(72) Inventors: Karla V. Ballman, New York, NY (US); E. Aubrey Thompson, Jacksonville, FL (US); Edith A. Perez, Ponte Vedra Beach, FL (US)

(73) Assignee: Mayo Foundation for Medical Education and Research, Rochester, MN (US)

(21) Appl. No.: 15/305,177

(22) PCT Filed: Apr. 20, 2015
(86) PCT No.: PCT/US15/26620

§ 371 Date: Oct. 19, 2016

Related U.S. Application Data
(60) Provisional application No. 61/982,251, filed on Apr. 21, 2014.

(51) Int. Cl. C12Q 1/68; A61K 31/337; A61K 39/395

(52) U.S. Cl. CPC C12Q 1/6886; A61K 39/3955; A61K 31/337; C12Q 2600/158; C12Q 2600/106; A61K 2039/505

(57) ABSTRACT

This document provides methods and materials involved in identifying mammals having breast cancer (e.g., HER2-positive breast cancer) responsive to trastuzumab as well as methods and materials involved in treating mammals having breast cancer (e.g., HER2-positive breast cancer) responsive to trastuzumab. For example, methods and materials for using expression level profiles to identify mammal having HER2-positive breast cancer with an increased likelihood of being responsive to trastuzumab are provided.

### CROSS REFERENCE TO RELATED APPLICATIONS

This application claims the benefit of U.S. Provisional Ser. No. 61/982,251 filed Apr. 21, 2014. This disclosure of the prior application is considered part of (and is incorporated by reference in) the disclosure of this application.

### BACKGROUND

1. Technical Field

This document relates to methods and materials involved in identifying mammals having breast cancer (e.g., HER2-positive breast cancer) responsive to trastuzumab as well as methods and materials involved in treating mammals having breast cancer (e.g., HER2-positive breast cancer). For example, this document provides methods and materials for using expression level profiles to identify a mammal as having breast cancer (e.g., HER2-positive breast cancer) responsive to trastuzumab.

2. Background Information

Clinical trials demonstrated the efficacy of trastuzumab in an adjuvant setting. 20-25 percent of patients with HER2-positive breast tumors, however, relapse despite HER2-targeted therapy. A number of potential mechanisms were proposed to account for differential response to HER2-targeted therapy, including overexpression of EGFR, cMYC, or ERBB3, mutational activation of PI3K, and mutational loss of PTEN (Arteaga et al., Nat. Rev. Clin. Oncol., 9(1):16-32 (2012)).

### SUMMARY

This document provides methods and materials involved in identifying mammals having breast cancer (e.g., HER2-positive breast cancer) responsive to trastuzumab as well as methods and materials involved in treating mammals having breast cancer (e.g., HER2-positive breast cancer) responsive to trastuzumab. For example, this document provides methods and materials for using expression level profiles to identify mammal having HER2-positive breast cancer with an increased likelihood of being responsive to trastuzumab. As described herein, the presence of an elevated level of expression of at least nine of the nucleic acids listed in Table 9 within a HER2-positive breast cancer sample from a mammal can indicate that that mammal (e.g., a human) has HER2-positive breast cancer with an increased likelihood of being responsive to trastuzumab. As also described herein, a mammal with breast cancer can be treated by detecting the presence of an elevated level of expression of at least nine of the nucleic acids listed in Table 9 within a HER2-positive breast cancer sample from a mammal and administering trastuzumab to that mammal.

Having the ability to identify mammals as having breast cancer (e.g., HER2-positive breast cancer) with an increased likelihood of being responsive to trastuzumab

as described herein can allow those breast cancer patients to be properly identified and treated in an effective and reliable manner. For example, the breast cancer treatments provided herein can be used to treat breast cancer patients identified as having breast cancer (e.g., HER2-positive breast cancer) with an increased likelihood of being responsive to trastuzumab.

In general, one aspect of this document features a method for identifying a mammal as having breast cancer with an increased likelihood of being responsive to trastuzumab. The method comprises, or consists essentially of, determining whether or not cancer cells from the mammal contain an elevated level of expression for at least nine of the nucleic acids listed in Table 9, wherein the presence of the elevated levels indicates that the mammal has breast cancer with an increased likelihood of being responsive to trastuzumab. The mammal can be a human. The elevated levels can be determined using a cDNA-mediated annealing, selection, extension, and ligation (DASL) assay. The breast cancer can be an HER2-positive breast cancer.

In another aspect, this document features a method for identifying a mammal as having breast cancer with an increased likelihood of being responsive to trastuzumab. The method comprises, or consists essentially of, (a) determining whether or not a breast cancer cells from the mammal contain an elevated level of expression for at least nine of the nucleic acids listed in Table 9, and (b) classifying the mammal as having breast cancer with an increased likelihood of being responsive to trastuzumab if the sample contains the elevated levels of the at least nine nucleic acids. The mammal can be a human. The elevated levels can be determined using a cDNA-mediated annealing, selection, extension, and ligation (DASL) assay. The breast cancer can be an HER2-positive breast cancer.

In another aspect, this document features a method for identifying a mammal as having breast cancer with an increased likelihood of being responsive to trastuzumab. The method comprises, or consists essentially of, (a) detecting the presence of an elevated level of expression for at least nine of the nucleic acids listed in Table 9 in breast cancer cells from the mammal, and (b) classifying the mammal as having breast cancer with an increased likelihood of being responsive to trastuzumab based at least in part on the presence of the elevated levels. The mammal can be a human. The elevated levels can be determined using a cDNA-mediated annealing, selection, extension, and ligation (DASL) assay. The breast cancer can be an HER2-positive breast cancer.

In another aspect, this document features a method for treating breast cancer. The method comprises, or consists essentially of, (a) detecting the presence of an elevated level of expression for at least nine of the nucleic acids listed in Table 9 in breast cancer cells from a mammal, and (b) administering a taxane compound and trastuzumab to the mammal under conditions wherein the number of breast cancer cells within the mammal is reduced. The mammal can be a human. The elevated levels can be determined using a cDNA-mediated annealing, selection, extension, and ligation (DASL) assay. The breast cancer can be an HER2-positive breast cancer. The taxane compound can be paclitaxel.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1: The N9831 multi-site phase III trial (NCT00005970) had three arms. Patients randomized to Arm A received doxorubicin and cyclophosphamide (AC) followed by weekly paclitaxel for 12 weeks (chemotherapy alone), whereas patients in Arms B and C received chemotherapy plus 12 months of trastuzumab. Arms B and C differed in that paclitaxel was given concurrently for the first month of trastuzumab treatment in Arm C, whereas trastuzumab was started after completion of paclitaxel therapy in Arm B. Women randomly assigned to the trastuzumab arms B and C had a significantly increased DFS (p<0.001) and overall survival (OS) (p<0.001) compared with women assigned to the control (chemotherapy alone) arm.

FIG. 2: Consort diagram describing the process whereby 1282 samples were selected for downstream analyses. The N9831 trial registered 3505 patients of whom 1282 (Arm A: 433, Arm B: 477, Arm C: 372) were evaluable for DASL gene expression profiling. The median follow-up time was 6 years, 11 months. All tumors included in this figure were tested for HER2 protein overexpression by immunohistochemistry (IHC) and/or gene amplification by fluorescent in situ hybridization (FISH) at a central laboratory (Mayo Clinic, Rochester, Minn.), and some tumors were excluded after central review of HER2 status. The largest cause of exclusion was insufficient tissue. Quality control (QC) failure after DASL analysis eliminated a small number of samples.

FIG. 3: Kaplan-Meier analysis of RFS in 1282 patients included in downstream analysis. In the N9831 comparison of sequential versus concurrent trastuzumab chemotherapy, there was an increase in DFS with concurrent trastuzumab (Arm C) compared to sequential trastuzumab (Arm B). Although outcome from the concurrent arm (Arm C) was slightly better than that from the sequential arm (Arm B), the significance did not cross the pre-specified O'Brien-Fleming boundary (p=0.00116) for the interim analysis of these two arms (Perez et al., J. Clin. Oncol., 29(34):4491-7 (2011)). The data shown in this figure indicate that outcome among the 1282 patients used to analyze gene expression recapitulates the outcome described elsewhere for all of the patients enrolled in N9831.

FIG. 4: Surface mapping reveals optimum values of q and m. A five-fold cross-validation (CV) using 100 iterations was used to identify the optimum values of q and m (number of m-genes with at least one probe above the q-quantile). For each of the 500 CV-iteration training sets, all values of m from 4 to 10 were paired with q-values from 0.25 to 0.75 by 0.01. The resulting 357 pairs of q/m values were used to determine enriched and not enriched tumors. Kaplan-Meier curves and log-rank tests were used to determine the hazard ratio and p-value for the difference between the arms for enriched tumors. Panel A shows the resulting contours of the HR and Panel B shows the p-values for one representative of the 500 CV-iterations. The optimum q/m pair was chosen via the minimum p-value. The dashed-lines in both panels show the HR and p-value for optimum q/m value for this CV-iteration.

FIG. 5: Network models reveal functional connections between genes associated with outcome in N9831. The Cytoscape Functional Interactome tool integrates functional relationships defined by multiple bioinformatics tools, including protein-protein and gene-gene interaction datasets. This tool was used to define networks associated with either decreased RFS (Panels A and C) or increased RFS (Panels B and D) in Arm A (Panels A and B) or Arms B/C (Panels C and D). Networks were constructed using genes with significant HRs (p<0.01), identified in Tables 4 and 5. Insertion of a single linker gene was allowed in network construction.

FIG. 6: A cohort of immune function genes is strongly associated with outcome after trastuzumab treatment, but has no effect on RFS following chemotherapy alone. Tumors in Arm A and Arms B/C were “binned” in to immune-enriched (IRE) and not immune-enriched (NIRE) using the voting model in which enrichment was defined by the m9q41 model. Panel A shows relapse-free survival (RFS) in years for enriched and not enriched subsets of tumors from both arms. Panel B shows relapse-free survival (RFS) in years for the enriched subset of tumors from both arms. Panel C shows relapse-free survival (RFS) in years for the non-enriched subset of tumors from both arms.

FIG. 7: Cross-validation of the immune function score model. The data were randomly split into 5 cohorts, and the optimal q/m combination was selected based on 4 cohorts. This q/m relationship was then used to determine whether a tumor was immune-enriched (IRE) or not enriched (NIRE) in the remaining cohort. Each tumor is classified 100 times (once for each cross-validation). The curves showed the results of the observed RFS based on these 100 different cross-validation sets, hence there are a total of n=128200 observations (Arm A.IRE 18117, Arm A.NIRE 25183, Arms B/C.IRE 36877, and Arms B/C.NIRE 48023).

### DETAILED DESCRIPTION

This document provides methods and materials involved in identifying mammals having breast cancer (e.g., HER2-positive breast cancer) responsive to trastuzumab as well as methods and materials involved in treating mammals having breast cancer (e.g., HER2-positive breast cancer) responsive to trastuzumab. For example, this document provides methods and materials for identifying a mammal as having HER2-positive breast cancer with an increased likelihood of being responsive to trastuzumab by determining whether or not a breast cancer sample from a mammal has an elevated level of expression for at least nine of the nucleic acids listed in Table 9. As described herein, if a mammal contains breast cancer cells (e.g., HER2-positive breast cancer cells) with an elevated level of expression for at least nine of the nucleic acids listed in Table 9, then that mammal can be classified as having HER2-positive breast cancer with an increased likelihood of being responsive to trastuzumab.

The term “elevated level” as used herein is in reference to the abundance of an individual mRNA in a given sample as compared to the abundance of that mRNA in a population of samples. A level is “elevated” when an mRNA abundance equals or is greater than 0.40 quantile for the population of samples for that specific mRNA. In general, the range of expression for the nucleic acids listed in Table 9 is defined for all tested samples and expressed as a range of 0 to 1.0 with 0 being the lowest and 1.0 being the highest quantile. The expression of each nucleic acid within a given sample is then referred to the distribution of expression within that population and defined as “elevated” when that expression level falls within the range of 0.40 to 1.0.

As described herein, the level of expression of nine or more of the nucleic acids listed in Table 9 within breast cancer cells can be used to determine whether or not a particular mammal has breast cancer (e.g., HER2-positive breast cancer) with an increased likelihood of being responsive to trastuzumab. Any appropriate breast cancer sample can be used as described herein to identify mammals having breast cancer (e.g., HER2-positive breast cancer) with an increased likelihood of being responsive to trastuzumab. For example, breast cancer tissue samples, breast cancer cell samples, and breast cancer needle biopsy specimen can be used to determine whether or not a mammal has breast cancer (e.g., HER2-positive breast cancer) with an increased likelihood of being responsive to trastuzumab. In addition, any appropriate method can be used to obtain breast cancer cells. For example, a breast cancer sample can be obtained by a tissue biopsy or following a surgical resection. Once obtained, a sample can be processed prior to measuring a level of expression. For example, a breast cancer sample can be processed to extract RNA from the sample. Once obtained, the RNA can be evaluated to determine the level of an mRNA of interest. In some cases, nucleic acids present within a sample can be amplified (e.g., linearly amplified) prior to determining the level of expression (e.g., using array technology). In another example, a breast cancer sample can be frozen, and sections of the frozen tissue sample can be prepared on glass slides. The frozen tissue sections can be stored (e.g., at −80° C.) prior to analysis, or they can be analyzed immediately (e.g., by immunohistochemistry with an antibody specific for a particular polypeptide of interest).

Any appropriate methods can be used to determine the level of expression of one or more of the nucleic acids listed in Table 9 within breast cancer cells. For example, quantitative real time PCR, in situ hybridization, or microarray technology can be used to determine whether or not a particular sample contains an elevated level of mRNA expression for a particular nucleic acid or lacks an elevated level of mRNA expression for a particular nucleic acid. In some cases, the level of expression can be determined using polypeptide detection methods such as immunochemistry techniques. For example, antibodies specific for FYN polypeptides can be used to determine the polypeptide level in a sample. In some cases, polypeptide-based techniques such as ELISAs and immunocytochemistry techniques can be used to determine whether or not a particular sample contains an elevated level of polypeptide expression for a particular nucleic acid or lacks an elevated level of polypeptide expression for a particular nucleic acid.

Once the levels of expression for at least nine of the nucleic acids listed in Table 9 within breast cancer cells from a mammal are determined, the levels can be compared to reference levels and used to classify the mammal as having or lacking breast cancer (e.g., HER2-positive breast cancer) with an increased likelihood of being responsive to trastuzumab as described herein.

This document also provides methods and materials for treating breast cancer (e.g., HER2-positive breast cancer). In some cases, a taxane compound (e.g., paclitaxel, Abraxane®, Taxol®, or docetaxel) and trastuzumab can be administered to a mammal (e.g., a human) having breast cancer (e.g., HER2-positive breast cancer) with an increased likelihood of being responsive to trastuzumab under conditions wherein the number of breast cancer cells or the progression of the breast cancer is reduced. For example, paclitaxel can be administered to a human having breast cancer at a dose of 80-100 mg/m² per week, while trastuzumab is administered to that same human at a dose of 2 mg/kg every week or 6 mg/kg every 3 weeks (after loading doses). In some cases, a non-taxane compound (e.g., eribulin, carboplatin, or vinorelbine) and trastuzumab can be administered to a mammal (e.g., a human) having breast cancer (e.g., HER2-positive breast cancer) with an increased likelihood of being responsive to trastuzumab under conditions wherein the number of breast cancer cells or the progression of the breast cancer is reduced.

In some cases, a mammal (e.g., a human) with breast cancer can be treated by detecting the presence of an elevated level of expression of at least nine of the nucleic acids listed in Table 9 within a HER2-positive breast cancer sample from a mammal and administering trastuzumab alone or combination with a taxane compound to that mammal.

The invention will be further described in the following examples, which do not limit the scope of the invention described in the claims.

### EXAMPLES

### Example 1

### Elevated Expression Levels of a Panel of Nucleic Acids can be Used to Identify Patients with Breast Cancer that is Responsive to Trastuzumab

### Patients

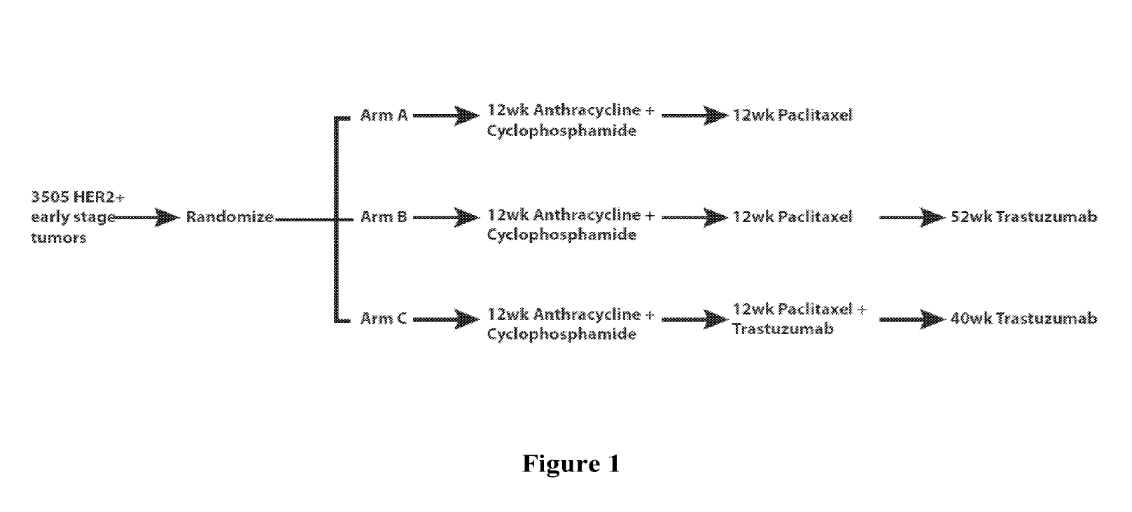

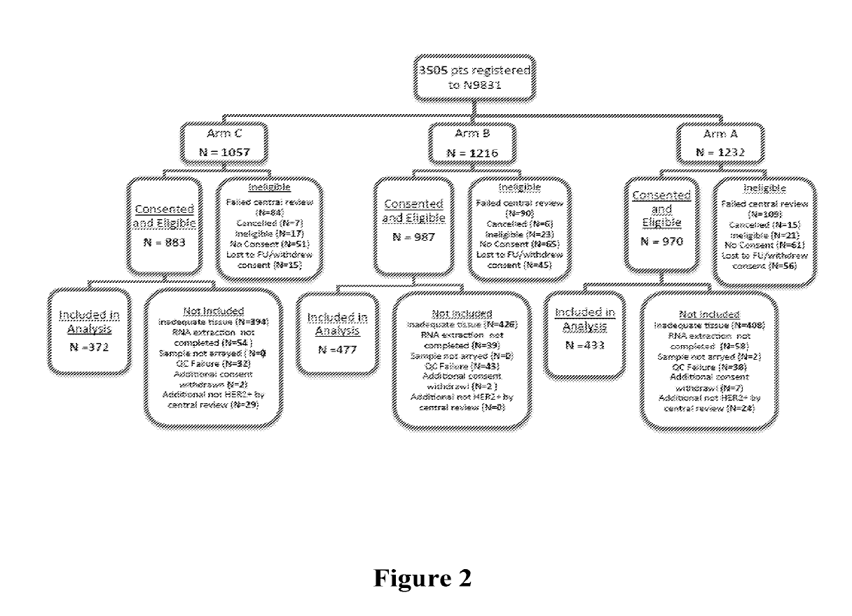

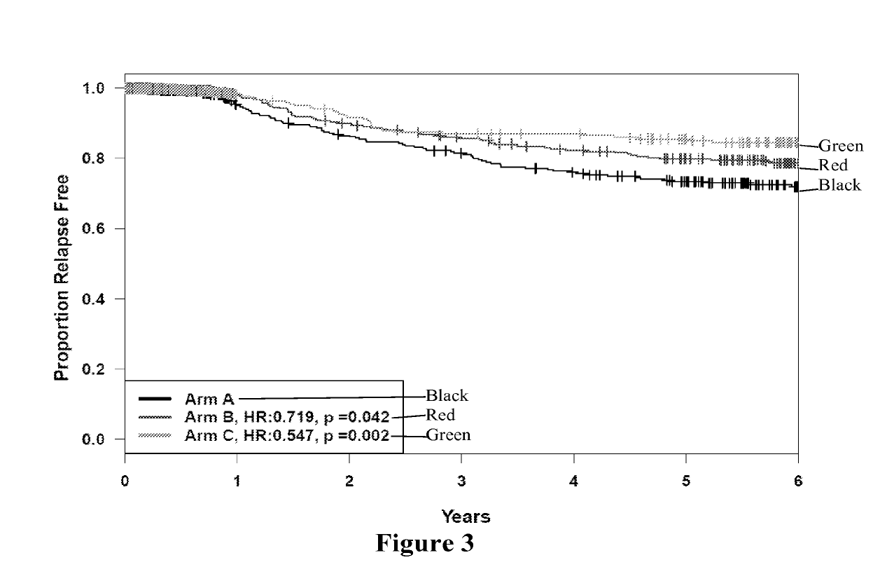

There were 3505 patients enrolled in N9831 (NCT 00005970) and randomized to 3 arms. All patients received anthracycline plus cyclophosphamide (AC). Arm A patients received paclitaxel alone; Arm B patients received paclitaxel followed by trastuzumab; and Arm C patients received paclitaxel and concurrent trastuzumab (FIG. 1) after completion of the AC therapy. From these patients, 1282 samples (Arm A-433, Arm B-477, Arm C-372) were evaluable for DASL gene expression profiling (FIG. 2). There were some differences in the clinical-pathological characteristics between the 1282 patients included in this analysis and the 2223 patients who were excluded (Table 1); however, the differences in outcomes among the three arms for the 1282 included patients (FIG. 3) were similar to those reported for the trial as a whole (Perez et al., J. Clin. Oncol., 29(25):3366-73 (2011)). Since the interest of this analysis is the biological basis that underlies trastuzumab response, the 849 patients who received trastuzumab (Arms B and C, denoted Arms B/C) were pooled.

TABLE 1
Patient demographics of 1282 samples included in the
DASL analysis vs. remaining patients registered on N9831.

| | DASL | | Remaining N9831 | | |
|---|---|---|---|---|---|
| | n = 1282 | | n = 2223 | | Chi-Square |
| Characteristic | No. | % | No. | % | P-Value |
| Age at Random | | | | | |
| Assignment, years | | | | | |
| 18-39 | 220 | 17.2 | 367 | 16.5 | 0.09* |
| 40-49 | 399 | 31.1 | 759 | 34.1 | |
| 50-59 | 425 | 33.2 | 716 | 32.2 | |
| ≧60 | 238 | 18.6 | 381 | 17.1 | |
| Extent of Surgery | | | | | |
| Mastectomy | 787 | 61.4 | 1361 | 61.2 | 0.26\hat{ } |
| Breast Spaning | 495 | 38.6 | 862 | 38.8 | |
| Extent of nodal | | | | | |
| examination | | | | | |
| Sentinel biopsy | 119 | 9.3 | 234 | 10.5 | 0.24\hat{ } |
| Axillary nodal | 1163 | 90.7 | 1989 | 89.5 | |
| dissection | | | | | |
| Tumor Size, cm | | | | | |
| ≦2.0 | 490 | 38.2 | 903 | 40.6 | 0.06* |
| 2.1-4.9 | 636 | 49.6 | 1092 | 49.1 | |
| ≧5.0 | 156 | 12.2 | 228 | 10.3 | |
| Histologically positive | | | | | |
| nodes | | | | | |
| 0 | 178 | 13.9 | 293 | 13.2 | 0.25* |
| 1-3 | 580 | 45.2 | 1094 | 49.2 | |
| 4-9 | 351 | 27.4 | 558 | 25.1 | |
| ≧10 | 173 | 13.5 | 278 | 12.5 | |
| Tumor grade | | | | | |
| 1 | 22 | 1.7 | 58 | 2.6 | 0.02*^{±} |
| 2 | 323 | 25.2 | 607 | 27.3 | |
| 3 | 924 | 72.1 | 1516 | 68.2 | |
| Unknown | 13 | 1 | 42 | 1.9 | |
| Estrogen receptor status | | | | | |
| Positive | 618 | 48.2 | 1223 | 55.0 | 0.009\hat{ }^{±} |
| Negative | 664 | 51.8 | 999 | 44.9 | |
| Unknown | 0 | 0.0 | 1 | 0.0 | |

*Mantel-Haenszel Chi-Square test
\hat{ }Chi-square test
^{±}Unknown not included in the statistical test.

Regarding Table 1, demographics of 1282 patients were included in downstream analyses. The clinical-pathological characteristics and outcomes of the 1282 patients enrolled on Arms A, B, and C reported herein were similar to those of the 2223 patients on Arms A, B, and C excluded from analysis. There was a small but significant increase in representation of ER-negative patients among those included for DASL analysis.

DASL Analysis of mRNA Abundance

Individual tumor blocks were examined microscopically, and tissue punches were obtained from demarcated areas of invasive tumor using a 1 mm biopsy punch with plunger (Fisher Scientific). Total RNA was extracted from at least one 1 mm tissue punch. Punches were deparaffinized in Citrisolv (Fisher Scientific) at room temperature for 30 minutes. The Citrisolv was aspirated, and the tissue was washed with 100% ethanol, vortexed, and centrifuged twice. Ethanol was removed, and the tissue was dried at 37° C. for 10 minutes. The samples were then incubated in Proteinase K Digestion (PKD) buffer and proteinase K (1 μg/μL) for overnight (at least 8 hours) at 56° C. The digested tissue was incubated for 15 minutes at 80° C. and centrifuged (14000 rpm) for 2 minutes at room temperature. The supernatant was collected, and the RNA extraction, including DNase I treatment, was completed using the RNeasy FFPE kit on an automated QIAcube platform according to the manufacturer's instructions (QIAGEN, Valencia, Calif.). The concentration of the purified RNA was determined using a NanoDrop ND-1000 spectrophotometer (Nanodrop Technologies; Wilmington, Del.). Purified total RNA was stored at −80° C. Labeling and hybridizations to BeadChips (HumanRef v4 Beadchip, Illumina) were performed as described elsewhere (Ton et al., Breast Cancer Research and Treatment, 125(3):879-83 (2011), Bibikova et al., Am. J. Pathol., 165(5):1799-807 (2004), Li et al., Cancer Res., 66(8):4079-88 (2006), and Reinholz et al., BMC Med. Genomics, 3:60 (2010)) with slight modifications. Samples (200 ng RNA) were randomized across 17 plates and subsequently to 136 chips according to date and order of RNA extraction, clinicopathologic characteristics, year on study, and treatment arm. The non-background corrected expression values from BeadStudio underwent a quality-control evaluation using the metrics of 1) proportion of probes detected at p<0.05, 2) inter-quartile range, and 3) skewness (Mahoney et al., BMC Res. Notes, 6(1):33 (2013)). In addition, a Stress metric, which quantified the amount of transformation that is required for an array to be normalized, was applied. The replicated patient sample with the lowest Stress value was used for analysis. Samples with a Stress value>log²(1.5) were deemed to be poor quality and removed. The remaining data were normalized using quantile normalization. A detailed description of the quality assessment protocols that were applied to these samples is described elsewhere (Mahoney et al., BMC Res. Notes, 6(1):33 (2013)).

Intra- and inter-plate technical replicates were performed using randomly selected N9831 patient samples and Universal Human Reference RNA (UHRR) control samples (Ambion Life Technologies). UHRR samples were analyzed in duplicate on every plate, with correlation coefficients of >0.9 for both UHRR and patient samples (Table 2 and Table 3, respectively), well within FDA and NCI guidelines that recommend CV values be less than 15% to be considered a precise assay.

TABLE 2
Replicate analyses using UHRR and patient samples indicates
a high degree of analytical precision on the DASL platform.
A. UHRR samples

| | | | Std | | | | |
|---|---|---|---|---|---|---|---|
| Comparison | N | Mean | Dev | L95 | IJ95 | Min | Max |
| Paired .1 vs .2 Samples | 17 | 0.9943 | 0.0062 | 0.9928 | 0.9958 | 0.9758 | 0.9986 |
| UHR01.1 vs Other .1 Samples | 16 | 0.9887 | 0.0075 | 0.9868 | 0.9906 | 0.9638 | 0.9948 |
| UHR02.1 vs Other .1 Samples | 16 | 0.9727 | 0.0067 | 0.971 | 0.9744 | 0.9602 | 0.9826 |
| UHR03.1 vs Other .1 Samples | 16 | 0.987 | 0.0037 | 0.9861 | 0.9879 | 0.9803 | 0.9938 |
| UHR04.1 vs Other .1 Samples | 16 | 0.9912 | 0.0054 | 0.9899 | 0.9925 | 0.9751 | 0.9966 |
| UHR05.1 vs Other .1 Samples | 16 | 0.9691 | 0.005 | 0.9876 | 0.9904 | 0.9752 | 0.9938 |
| UHR06.1 vs Other .1 Samples | 16 | 0.9662 | 0.006 | 0.9847 | 0.9877 | 0.9764 | 0.995 |
| UHR07.1 vs Other .1 Samples | 16 | 0.9912 | 0.0041 | 0.9902 | 0.9922 | 0.98 | 0.9958 |
| UHR08.1 vs Other .1 Samples | 16 | 0.9905 | 0.0061 | 0.989 | 0.992 | 0.9718 | 0.996 |
| UHR09.1 vs Other .1 Samples | 16 | 0.9923 | 0.0039 | 0.9913 | 0.9933 | 0.9796 | 0.9956 |
| UHR10.1 vs Other .1 Samples | 16 | 0.9927 | 0.0059 | 0.9912 | 0.9942 | 0.9726 | 0.9973 |
| UHR11.1 vs Other .1 Samples | 16 | 0.9912 | 0.0073 | 0.9894 | 0.993 | 0.967 | 0.9967 |
| UHR12.1 vs Other .1 Samples | 16 | 0.9894 | 0.0037 | 0.9885 | 0.9903 | 0.9826 | 0.9947 |
| UHR13.1 vs Other .1 Samples | 16 | 0.9918 | 0.0057 | 0.9904 | 0.9932 | 0.9732 | 0.9973 |
| UHR14.1 vs Other .1 Samples | 16 | 0.9903 | 0.006 | 0.9888 | 0.9918 | 0.9738 | 0.9966 |
| UHR15.1 vs Other .1 Samples | 16 | 0.9916 | 0.0064 | 0.99 | 0.9932 | 0.9699 | 0.9968 |
| UHR16.1 vs Other .1 Samples | 16 | 0.9892 | 0.0083 | 0.9871 | 0.9913 | 0.9625 | 0.9975 |
| UHR17.1 vs Other .1 Samples | 16 | 0.9875 | 0.009 | 0.9853 | 0.9897 | 0.9602 | 0.9975 |
| UHR01.2 vs Other .2 Samples | 16 | 0.985 | 0.0038 | 0.984 | 0.966 | 0.9791 | 0.9928 |
| UHR02.2 vs Other .2 Samples | 16 | 0.9908 | 0.0033 | 0.99 | 0.9916 | 0.9833 | 0.9951 |
| UHR03.2 vs Other .2 Samples | 16 | 0.978 | 0.0058 | 0.9766 | 0.9794 | 0.9716 | 0.9928 |
| UHR04.2 vs Other .2 Samples | 16 | 0.992 | 0.0051 | 0.9907 | 0.9933 | 0.977 | 0.9965 |
| UHR05.2 vs Other .2 Samples | 16 | 0.987 | 0.0054 | 0.9857 | 0.9883 | 0.9756 | 0.9943 |
| UHR06.2 vs Other .2 Samples | 16 | 0.9876 | 0.0051 | 0.9863 | 0.9889 | 0.976 | 0.9939 |
| UHR07.2 vs Other .2 Samples | 16 | 0.9908 | 0.003 | 0.99 | 0.9916 | 0.9845 | 0.9951 |
| UHR08.2 vs Other .2 Samples | 16 | 0.9908 | 0.0052 | 0.9895 | 0.9921 | 0.9763 | 0.9965 |
| UHR09.2 vs Other .2 Samples | 16 | 0.9926 | 0.004 | 0.9916 | 0.9936 | 0.9803 | 0.9973 |
| UHR10.2 vs Other .2 Samples | 16 | 0.9925 | 0.0058 | 0.9911 | 0.9939 | 0.9756 | 0.9974 |
| UHR11.2 vs Other .2 Samples | 16 | 0.9919 | 0.0058 | 0.9904 | 0.9934 | 0.9748 | 0.9974 |
| UHR12.2 vs Other .2 Samples | 16 | 0.9917 | 0.0033 | 0.9909 | 0.9925 | 0.9819 | 0.9973 |
| UHR13.2 vs Other .2 Samples | 16 | 0.9908 | 0.0058 | 0.9893 | 0.9923 | 0.9738 | 0.9967 |
| UHR14.2 vs Other .2 Samples | 16 | 0.9885 | 0.0069 | 0.9868 | 0.9902 | 0.9716 | 0.9958 |
| UHR15.2 vs Other .2 Samples | 16 | 0.9912 | 0.0063 | 0.9896 | 0.9926 | 0.9732 | 0.9974 |
| UHR16.2 vs Other .2 Samples | 16 | 0.9888 | 0.0067 | 0.9871 | 0.9905 | 0.9722 | 0.9969 |
| UHR17.2 vs Other .2 Samples | 16 | 0.9886 | 0.0063 | 0.987 | 0.9902 | 0.9728 | 0.9969 |

TABLE 3
B. Patient (FFPE) samples

| | Different | Same |
|---|---|---|
| Duplicates | arrays | array |
| t0054.1 vs. t0054.3 | 0.950657 | 0.951242 |
| t0085.1 vs. t0085.3 | 0.910656 | 0.928123 |
| t0121.1 vs. t0121.3 | 0.941182 | 0.944025 |
| t0213.1 vs. t0213.3 | 0.956316 | 0.95884 |
| t0329.1 vs. t0329.3 | 0.951724 | 0.950645 |
| t0495.1 vs. t0495.3 | 0.933328 | 0.940598 |
| t0566.1 vs. t0566.3 | 0.868778 | 0.927531 |
| t0601.1 vs. t0601.3 | 0.865637 | 0.851786 |
| t0603.1 vs. t0603.3 | 0.914319 | 0.948114 |
| t0731.1 vs. t0731.3 | 0.86959 | 0.883358 |
| t0824.1 vs. t0824.3 | 0.904838 | 0.917697 |
| t0828.1 vs. t0828.3 | 0.908614 | 0.906578 |
| t0833.1 vs. t0833.3 | 0.937533 | 0.940288 |
| t0927.1 vs. t0927.3 | 0.905713 | 0.937256 |
| t0973.1 vs. t0973.3 | 0.868624 | 0.863294 |
| t1096.1 vs. t1096.3 | 0.748297 | 0.734954 |
| t1115.1 vs. t1115.3 | 0.83528 | 0.870667 |
| t1199.1 vs. t1199.3 | 0.950696 | 0.956579 |
| t1250.1 vs. t1250.3 | 0.918797 | 0.945575 |
| t1371.1 vs. t1371.3 | 0.94303 | 0.936145 |
| t1377.1 vs. t1377.3 | 0.903578 | 0.908383 |
| t1443.1 vs. t1443.3 | 0.943752 | 0.966925 |
| t1500.1 vs. t1500.3 | 0.847787 | 0.864959 |

Regarding Tables 2 and 3, pairwise Spearman rank correlation coefficients of quintile-normalized, log 2 transformed data from 34 UHRR samples (identified as UHR01-UHR17, with duplicates assayed on different plates designated UHR.1 vs UHR.2) were analyzed. Table 2 shows correlation coefficients for all samples against all other samples. The correlation coefficients for duplicate samples on the same plate averaged 0.994 (S.D.=0.006, 95% C.I. 0.98-1.00, range 0.97-1.0), whereas correlation coefficients for duplicates run on separate plates averaged 0.989 (S.D.=0.006, 95% C.I. 0.989-0.990. range 0.97-1.0). Likewise, 23 duplicate FFPE patient samples were analyzed in duplicate on the same plate and twice on two different plates (Table 3). The correlation coefficients for patient samples run in duplicate (identified by 0.1 and 0.3) on the same plate averaged 0.91 (S.D.=0.052, 95% C.I. 0.74-0.97, range 0.73-0.97), whereas correlations for duplicate patient samples run on two different plates averaged 0.903 (S.D.=0.05 95% C.I. 0.75-0.96, range 0.75-0.96).

TABLE 4
Genes with significant adjusted HRs in Arm A.

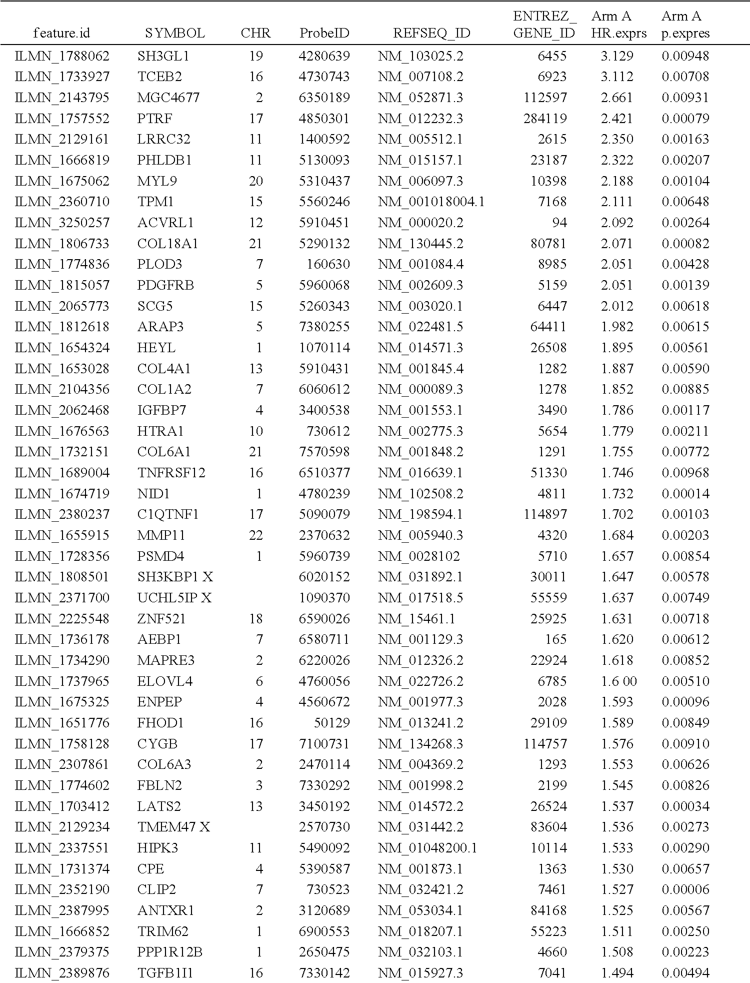

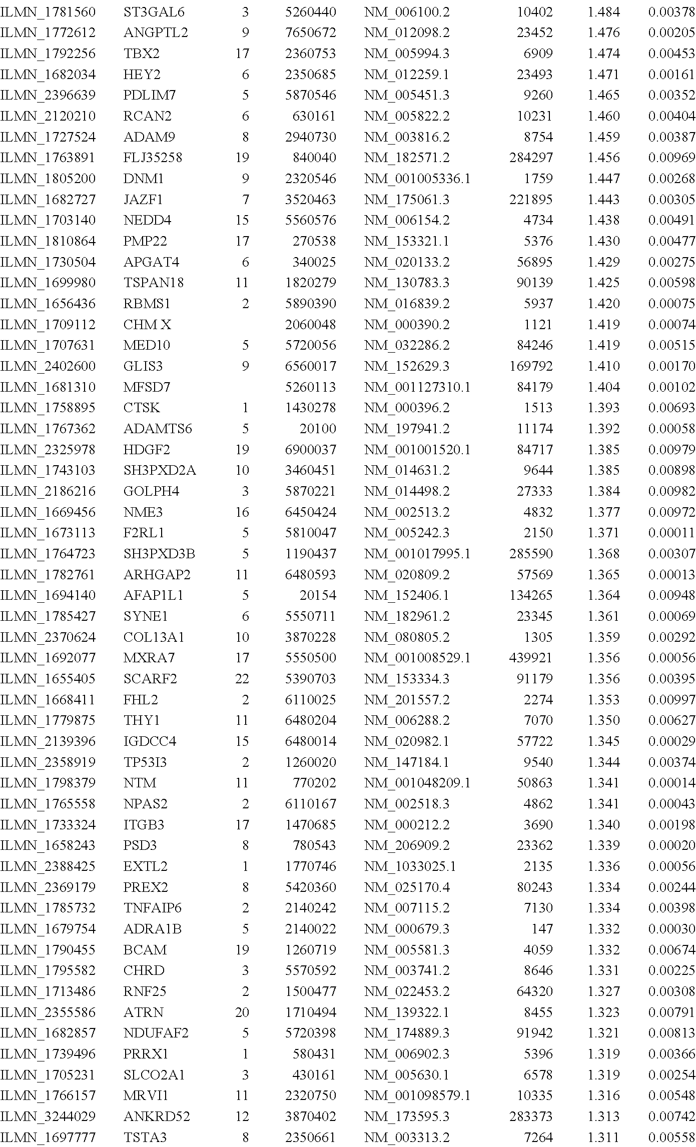

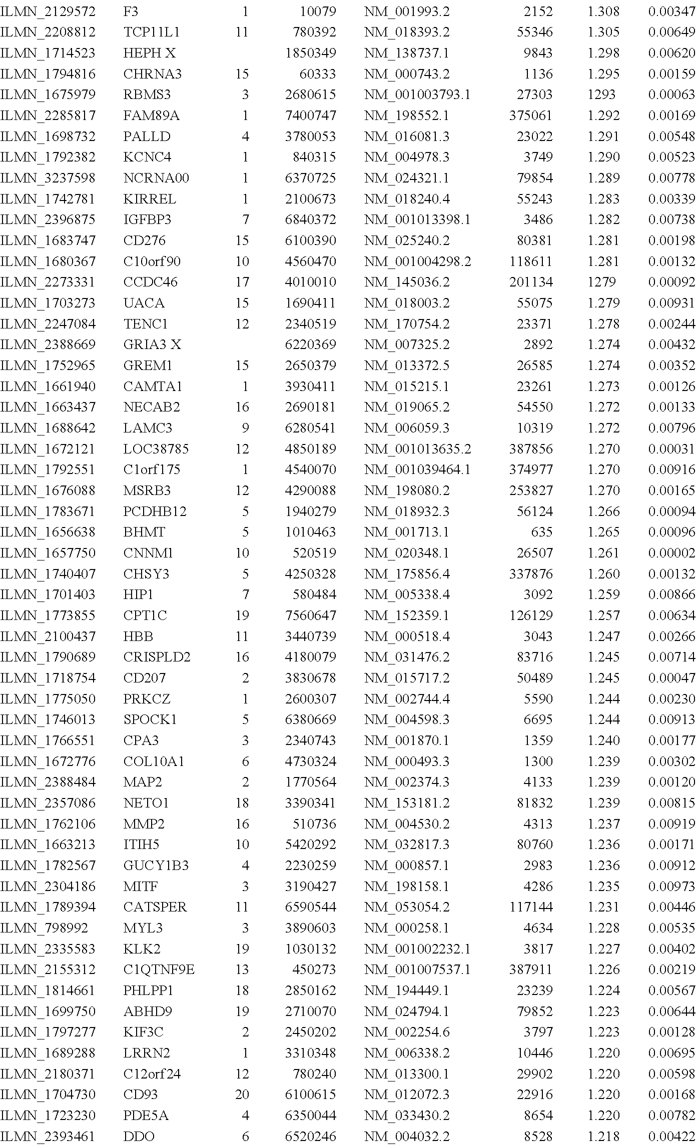

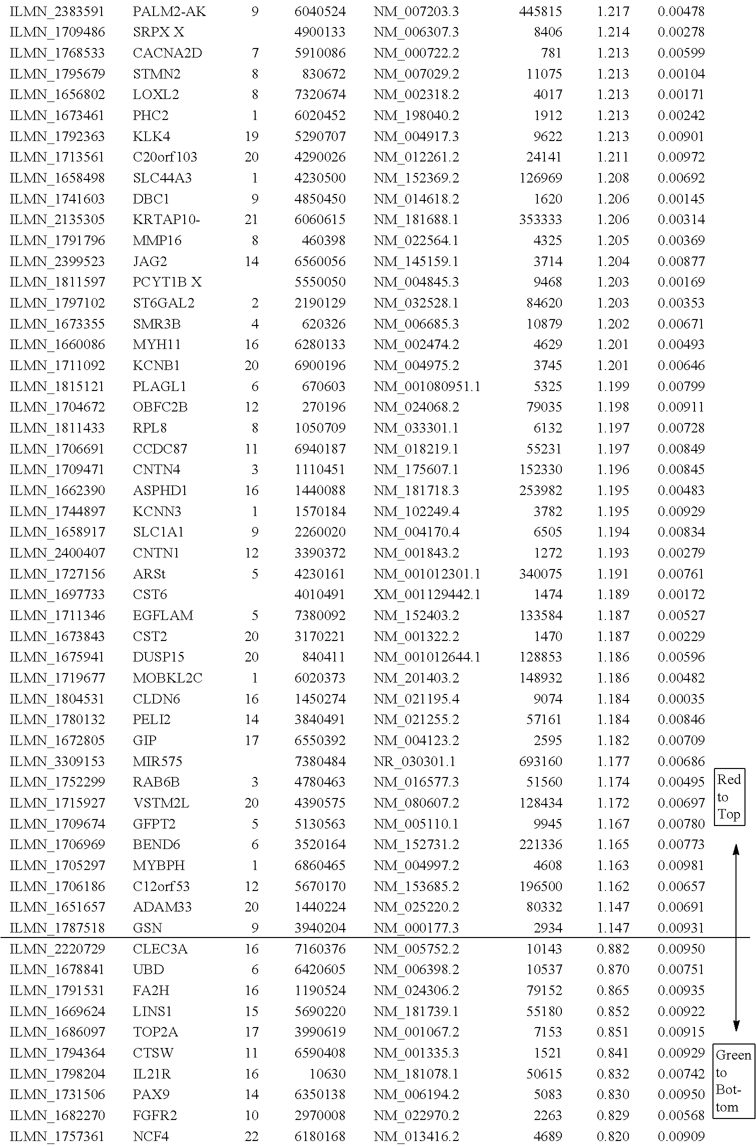

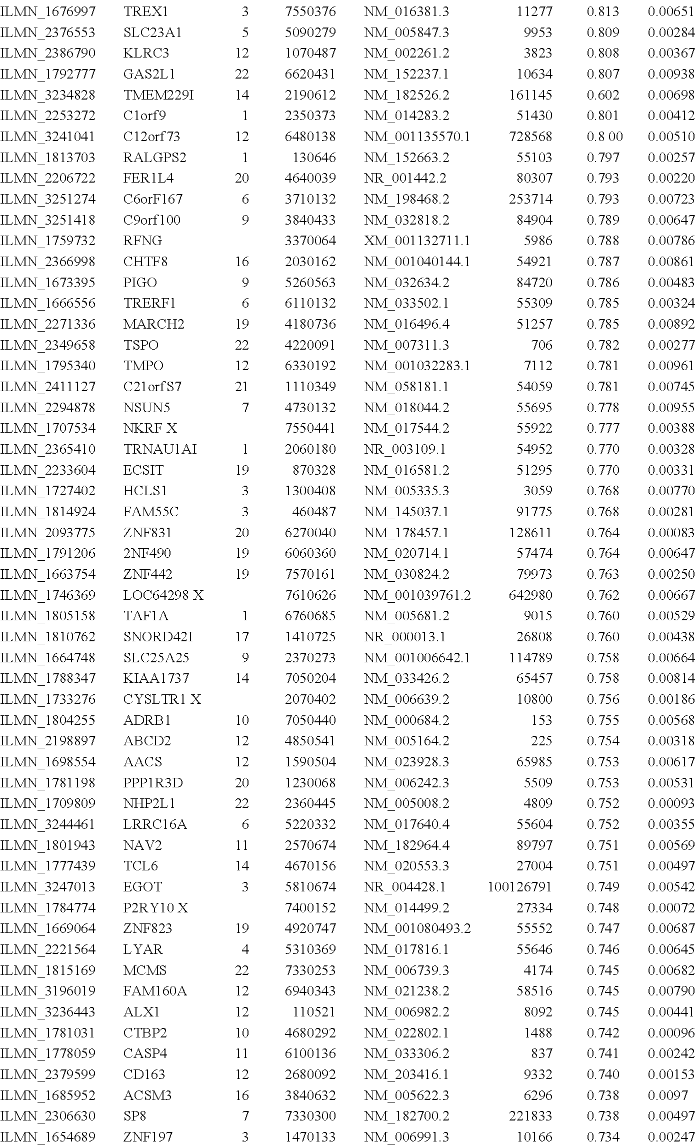

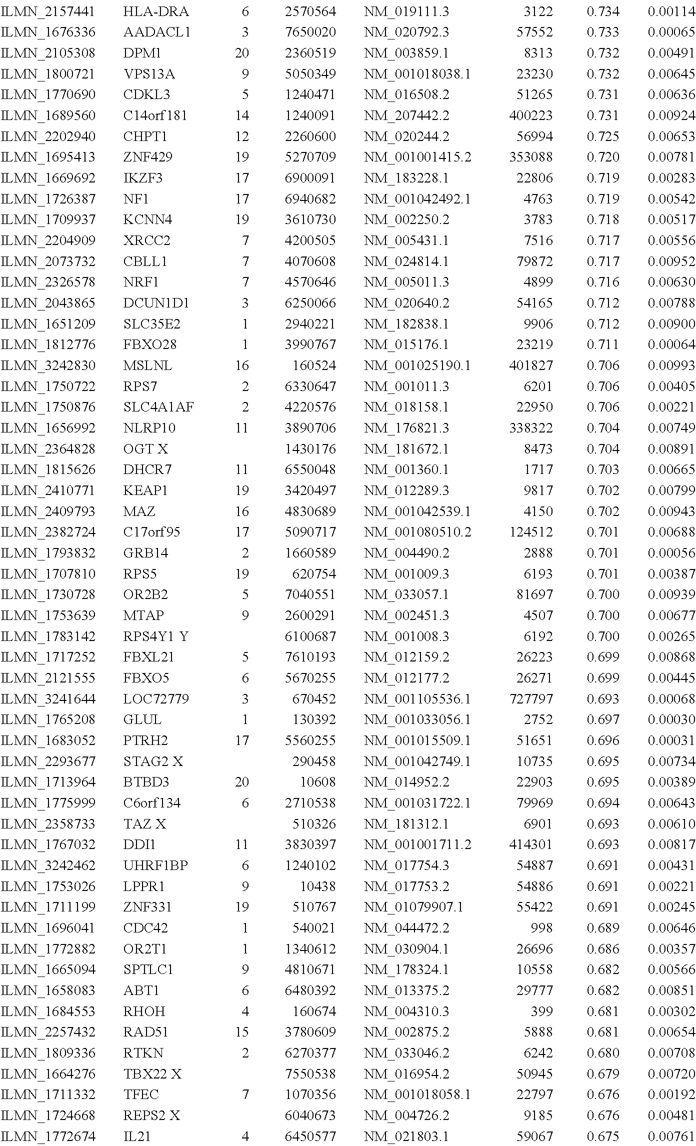

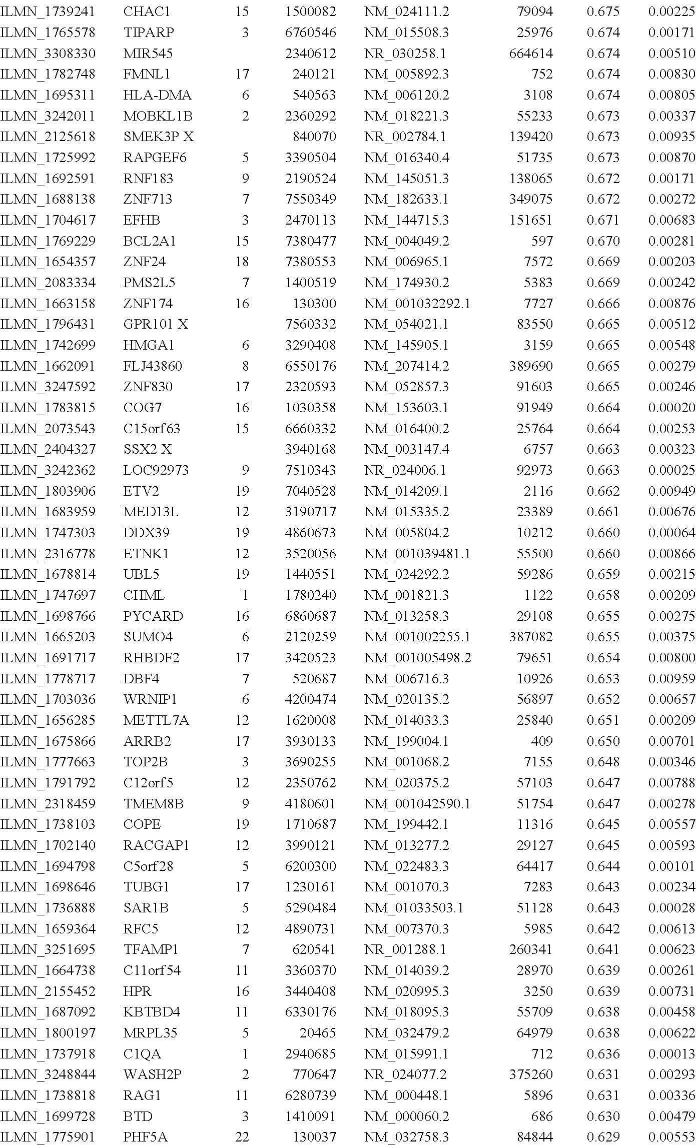

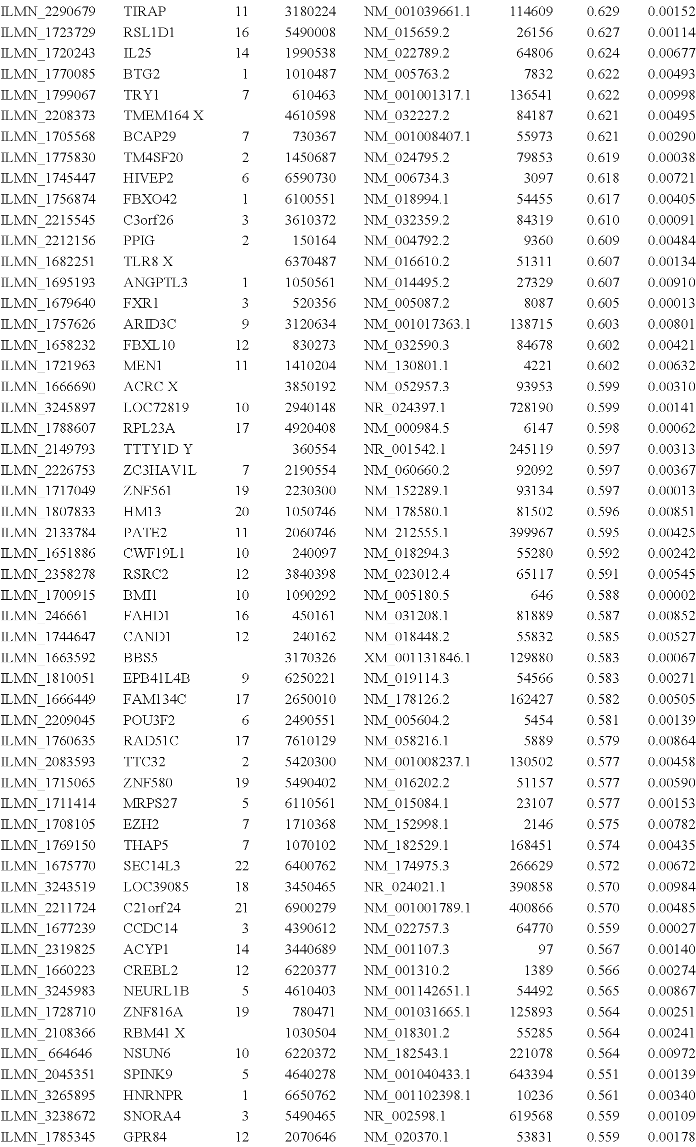

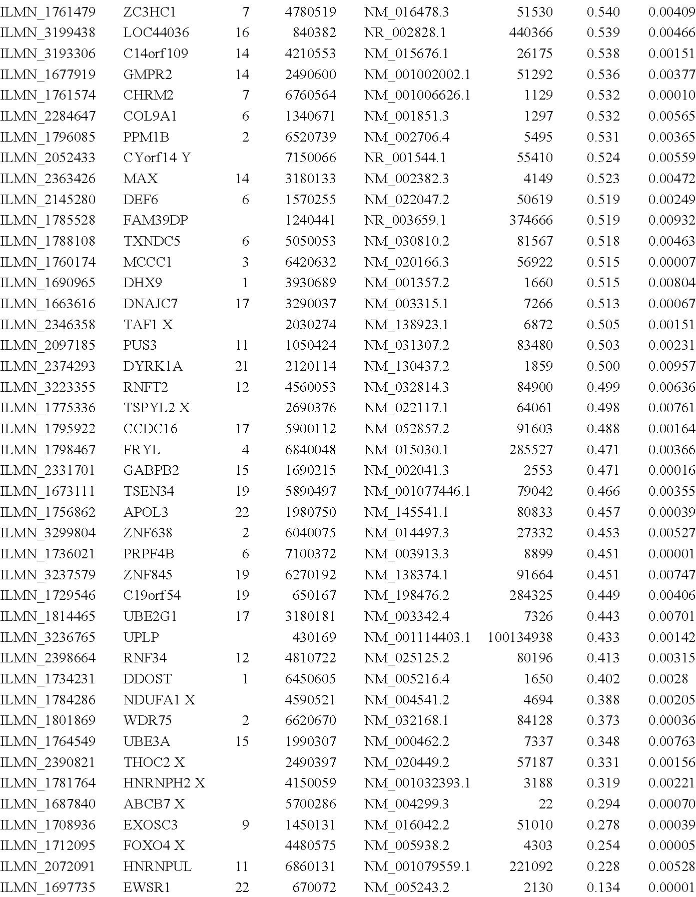

Regarding Table 4, genes with adjusted HRs>1 (p<0.01) are shown in the top section, whereas genes with HRs<1 (p<0.01) are shown in bottom section. CoxPH analysis (adjusted for significant clinical/pathological variables) was carried out using gene expression data from the DASL arrays and RFS as a continuous variable. Filtering was conducted to identify probes which had a median expression across all arms that were above the lowest 20% and below the highest 2%.

### Statistical Analysis of Cox Hazard Ratios (HR)

The primary endpoint was relapse-free survival (RFS), which was defined as the time from randomization to first local, regional, or distant recurrence, or the development of a new contralateral primary breast cancer. Multivariable Cox models (adjusting for nodal status, tumor size, hormone receptor status, age, and tumor grade) were used to evaluate the association between RFS and probe expression for all genes. The association was assessed separately within each patient group to understand biological processes that might be involved with response to trastuzumab. Probes meeting the filtering criteria and having an adjusted-model p<0.01 were considered to be significantly associated with RFS for the purpose of the functional analysis. Cox proportional models, which included the prognostic factors listed above as adjusting variables, were evaluated on the set of all patients and included probe, treatment group, and probe-treatment group interaction terms to identify probes that were potentially predictive of trastuzumab response.

### Functional Analysis

Cox hazard ratios were determined for all genes from the DASL analysis using time to event (RFS) as a continuous variable, as described herein. The Cytoscape Functional Interactome tool (Matthews et al., Nucleic Acids Res., 37(Database issue):D619-22 (2009)) was used to define networks associations among genes with Cox hazard ratios with adjusted-model p<0.01. Functional processes associated with network components were deduced from the pathway enrichment statistics function within the Cytoscape Functional Interactome tool.

### Enrichment of Gene Ontology Biological Process Terms

Functional ontology enrichment was determined by analysis of Gene Ontology Biological Process (GO:BP) terms using Fisher's exact test. Individual GO terms apply to many genes, and individual genes may have many associated GO terms. This one-to-many relationship between genes and Gene Ontology (GO) terms was downloaded from the BioMart portal at Ensembl (http://useast.ensembl.org/biomart/martview/). The Ensembl human gene annotation version 70 (v70) was used to identify genes. A developed script was used to assign each gene into all possible GO terms to which it belongs. This was done on both the genes with significant hazard ratios (HR), as well as all genes in the v70 annotation. For each of the GO terms, a Fisher's exact test was performed on a two-by-two contingency table with: (1) the number of genes with significant HR belonging to the GO term from Arm A; (2) the number of genes with significant HR belonging to the GO term from Arms B/C; (3) the numbers of genes, excluding those in (1), from all v70 genes that assigned to the GO term; and (4) the numbers of genes, excluding those in (2), from all v70 genes that assigned to the GO Term.

### Statistical Analysis

A decision was made not to split the samples into separate training and validation sets for the signature development due to the limited power in the overall dataset (204 recurrence events, with 89 in Arm A and 115 in Arms B/C). A split-sample approach, in which the data are divided into two cohorts for training and validation, fails to use all the information in the sample for signature development, yielding a noisy signature (Subramanian and Simon, Stat. Med., 30(6):642-53 (2011)). For a preliminary validation of the signature, cross-validation was used as described below.

The analyses focused on genes that had a plausible biological function with respect to trastuzumab response, as identified by network and functional ontology analysis. A voting scheme was used to develop a signature from a cohort of genes with HR<1.0, adjusted-model p<0.01, and interaction p<0.05. Since it is likely that the contribution of individual genes within the biological process might vary from tumor to tumor, a voting scheme was used to develop a signature. A tumor was designated as enriched for a biological function if m or more of the genes in the functional group had one or more probes expressed above a quantile q threshold. To determine the best pair of m and q values, a response surface was searched that consisted of all quantile values of q, between 0.25 and 0.75 by increments of 0.01. For each q/m pair, tumors were classified as enriched if they had m or more genes with at least one probe having an expression value above the q quantile for that probe across all samples. The q/m pair that was selected as best had the smallest p-value for a comparison of RFS between women with enriched tumors (as determined by the voting scheme based on q/m values) who were treated with trastuzumab compared to women with enriched tumors that were not treated with trastuzumab.

### Cross-Validation of the Signature Development

A cross-validation method was used to assess whether the observed predictive nature of the signature was generalizable. Since the feature selection was based on identified biological processes that differed between Arms A and B/C, it was not possible to do a complete cross-validation of the entire process starting from feature selection. However, the development of the signature was cross-validated based on the selected probes.

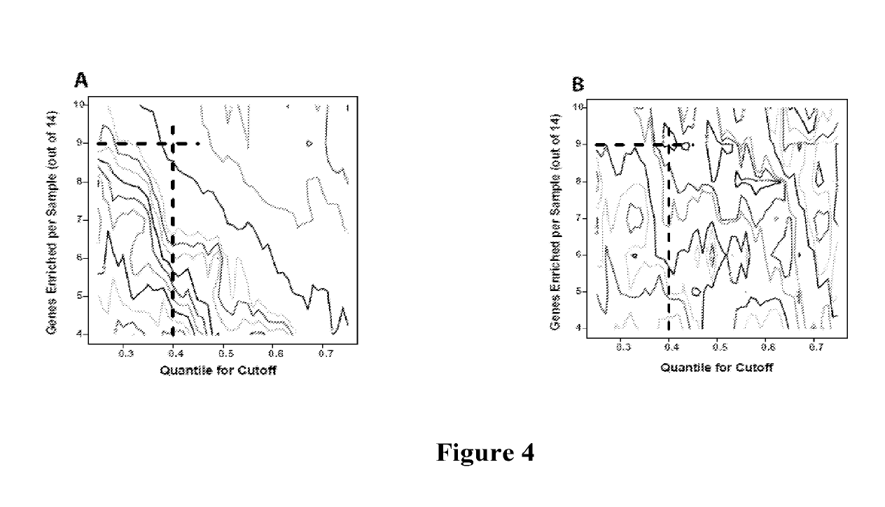

A five-fold cross validation was replicated 100 times for determining the performance of the voting scheme for classifying tumors as enriched or not enriched and whether the resulting signature appears predictive of RFS. During each cross validation replicate, all patients were randomly assorted into five different cohorts. Four of the cohorts were then used to define the best set of q/m pairs, searching the q/m grid (FIG. 4). The q/m pairs determined in this fashion were then used to define the immune enrichment scores of the “left out” 1/5 of the tumors. This procedure was repeated five times leaving out one of the cohorts each time. Replicating this analysis 100 times determined each tumor as immune enriched or not-enriched.

### Final Voting Scheme Values and Analysis

Using the selected q/m values, patients were grouped into enriched and non-enriched groups. Kaplan-Meier curves were used to summarize the RFS for each group and compared with a logrank test. Multivariable Cox models adjusted for the prognostic factors (listed above) and with treatment group, enriched status (determined by the voting scheme), and the treatment group-enriched status interaction term were used to determine whether the signature was potentially predictive.

### Gene Expression and Outcome Association

Multivariable Cox regression was used to identify genes significantly associated with RFS in Arm A and Arms B/C. 473 genes were identified that were associated with RFS at adjusted-model p<0.01 in Arm A (Table 4). We identified 510 genes significantly associated with RFS at adjusted-model p<0.01 in Arms B/C (Table 5).

TABLE 5
Genes with significant adjusted HRs in Arms B/C (chemotherapy plus trastuzumab) of N9831.

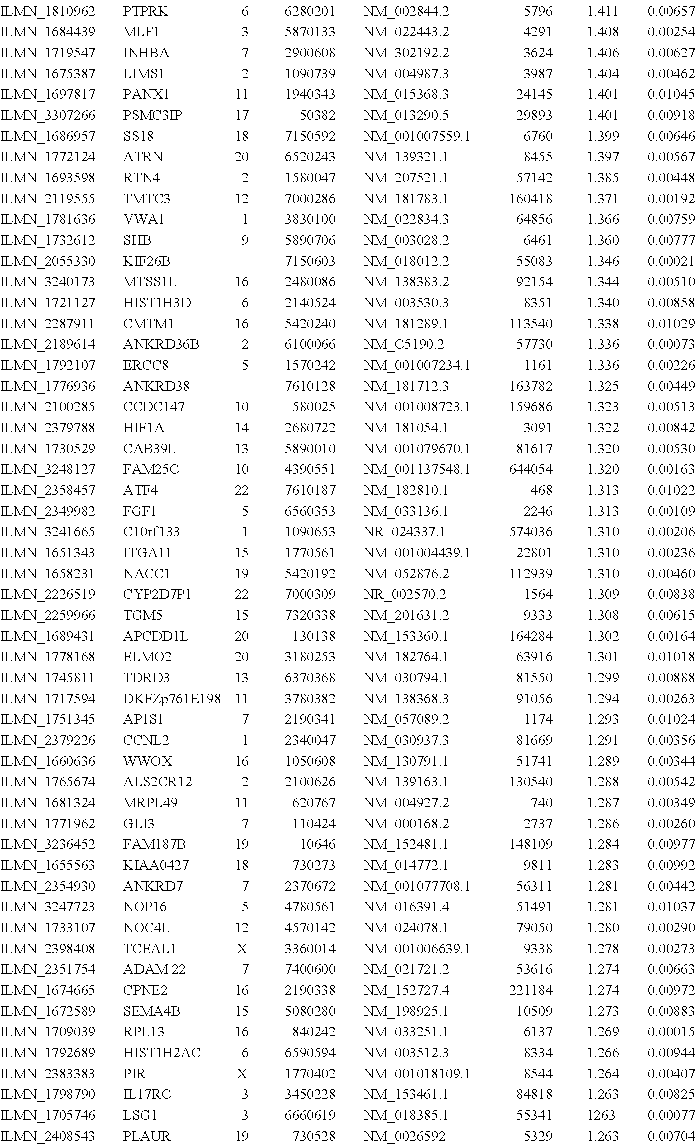

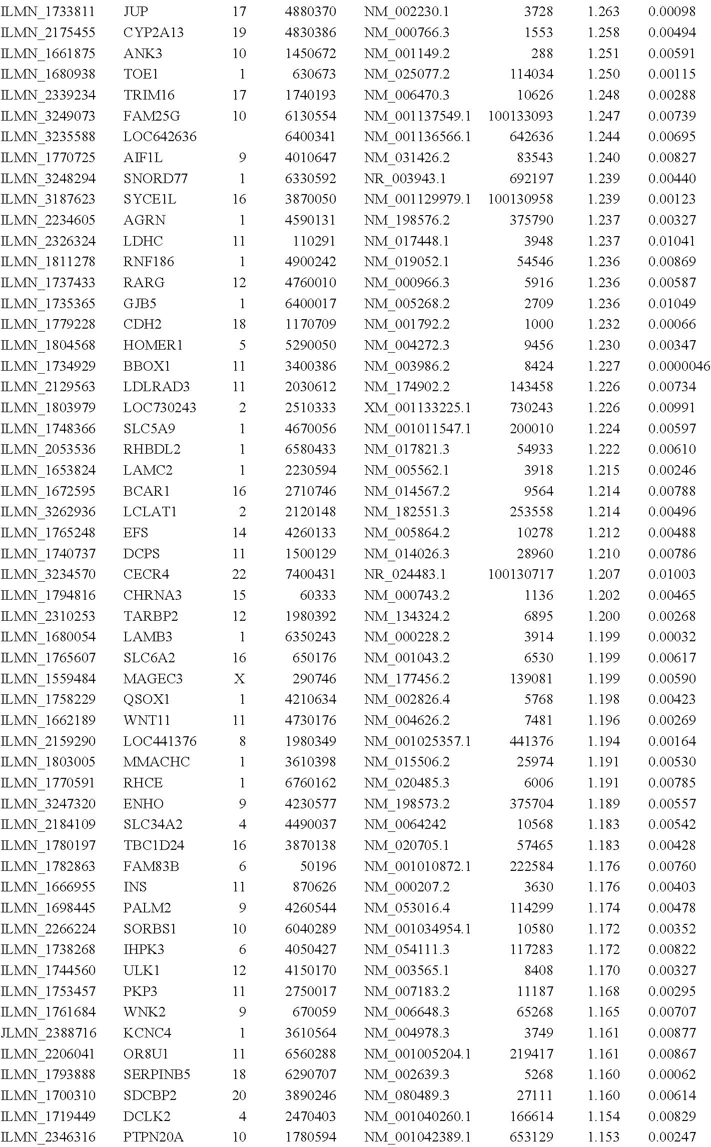

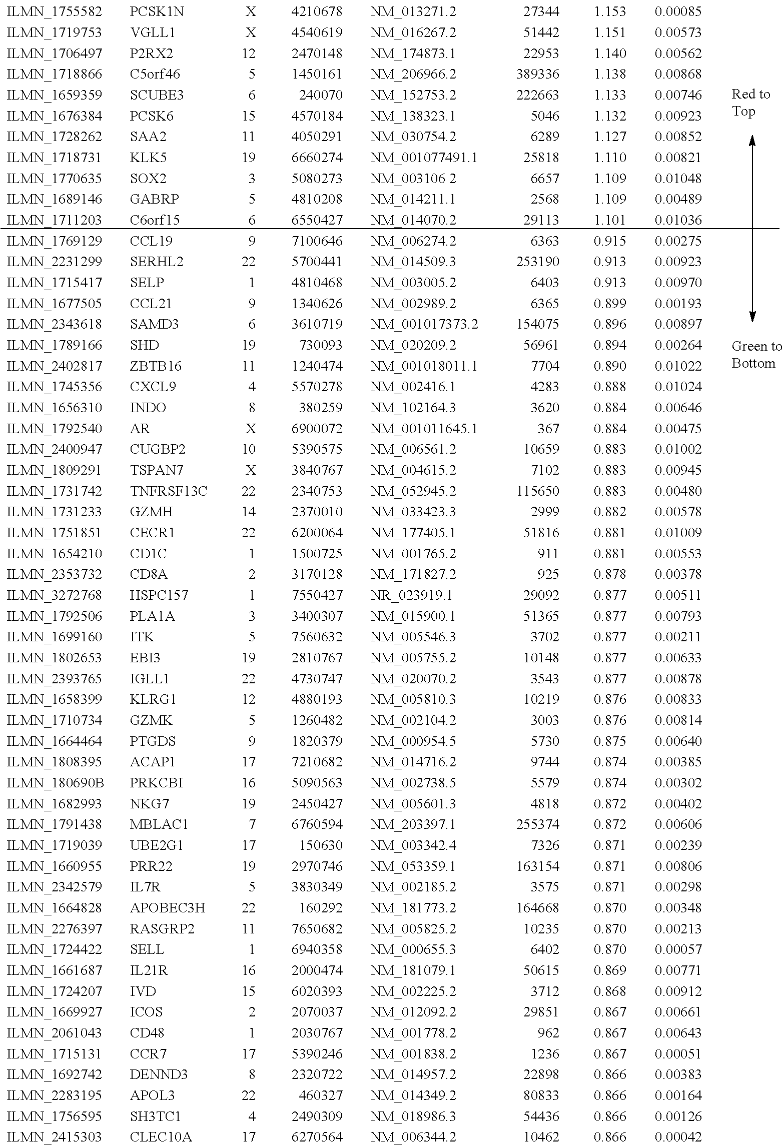

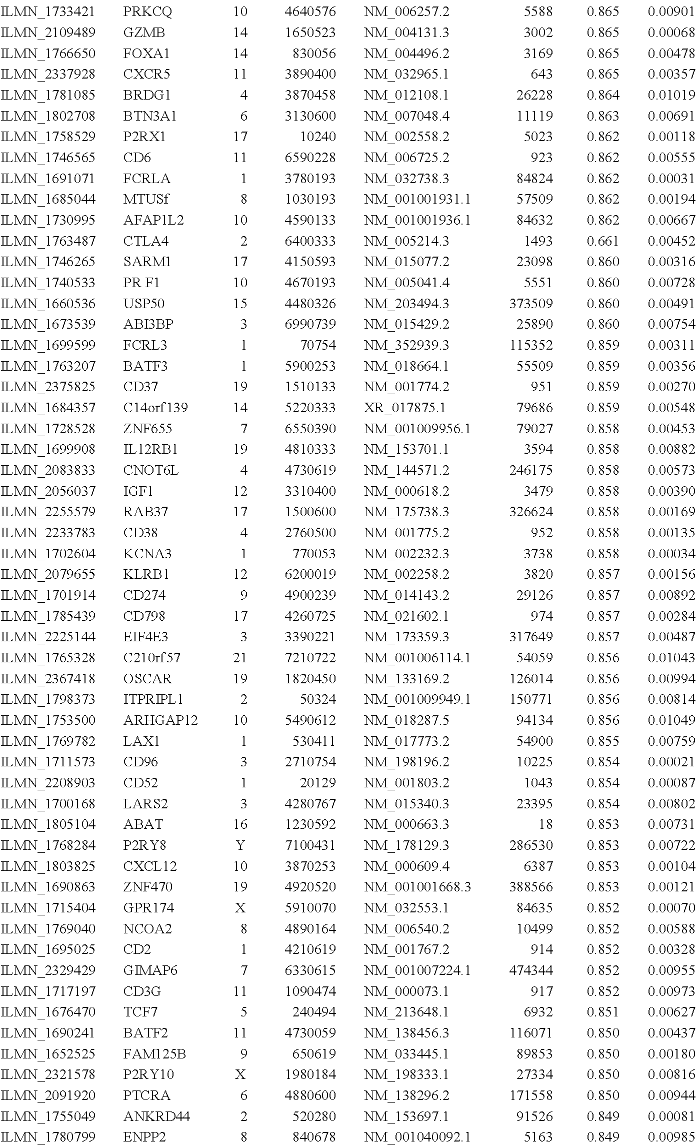

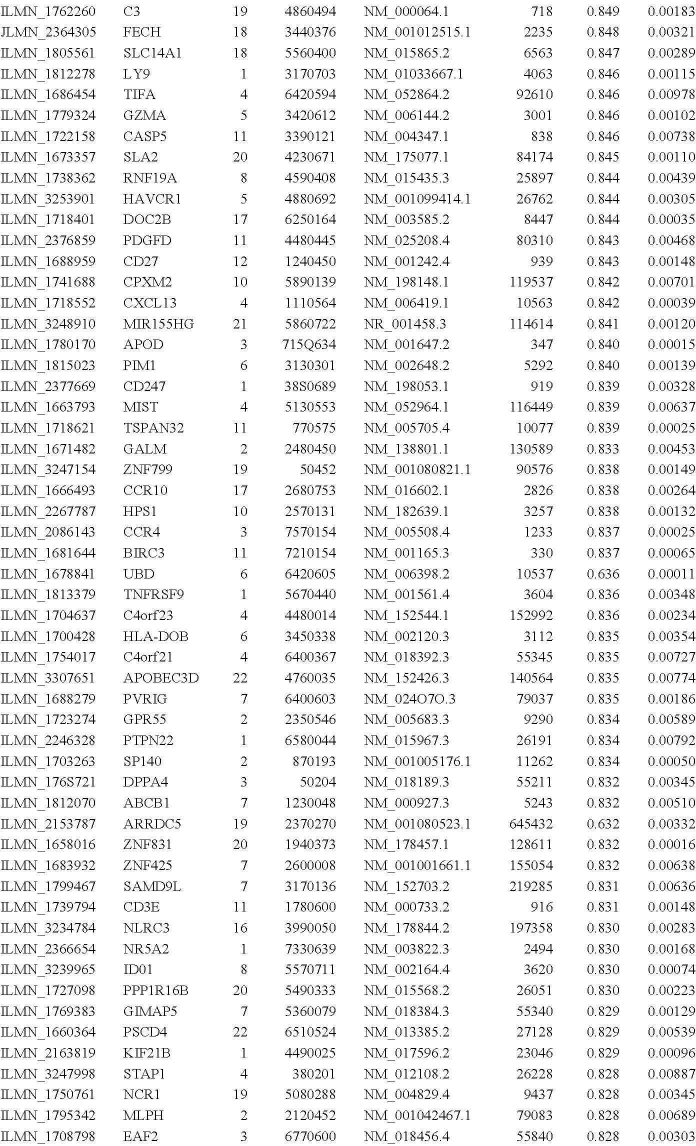

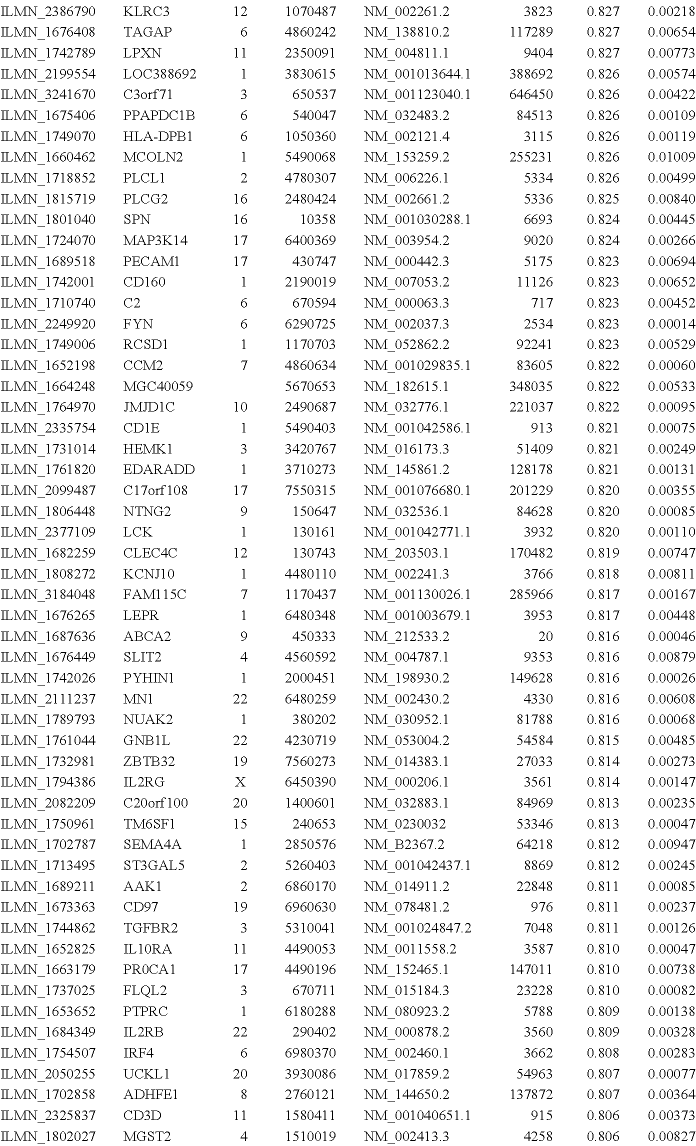

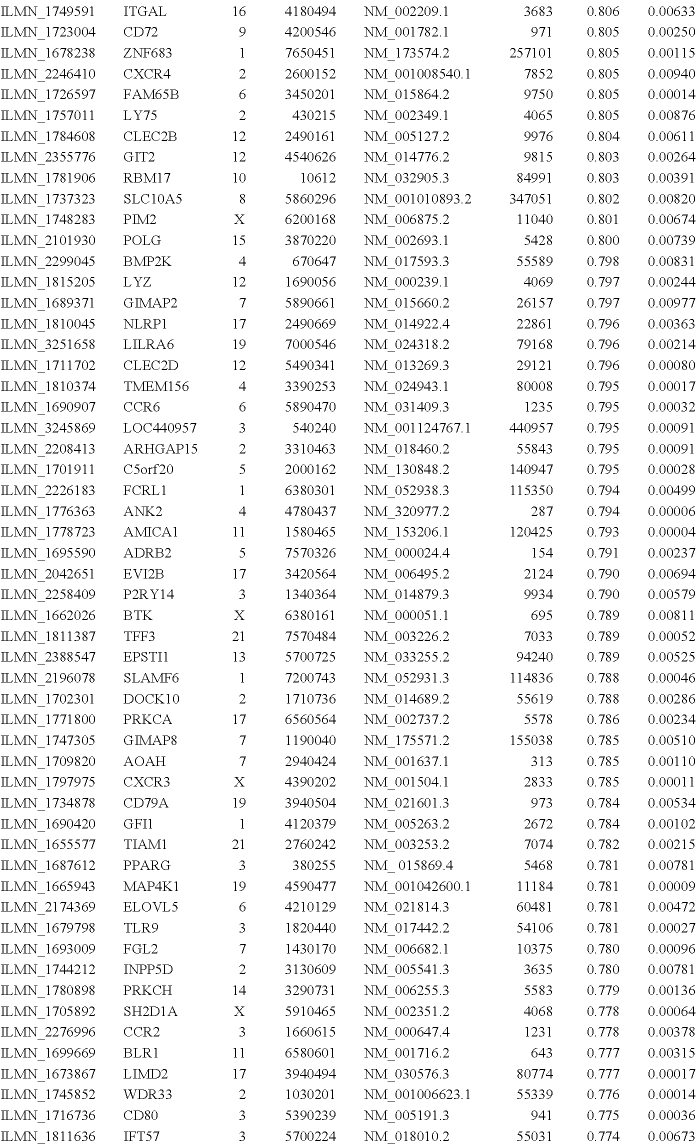

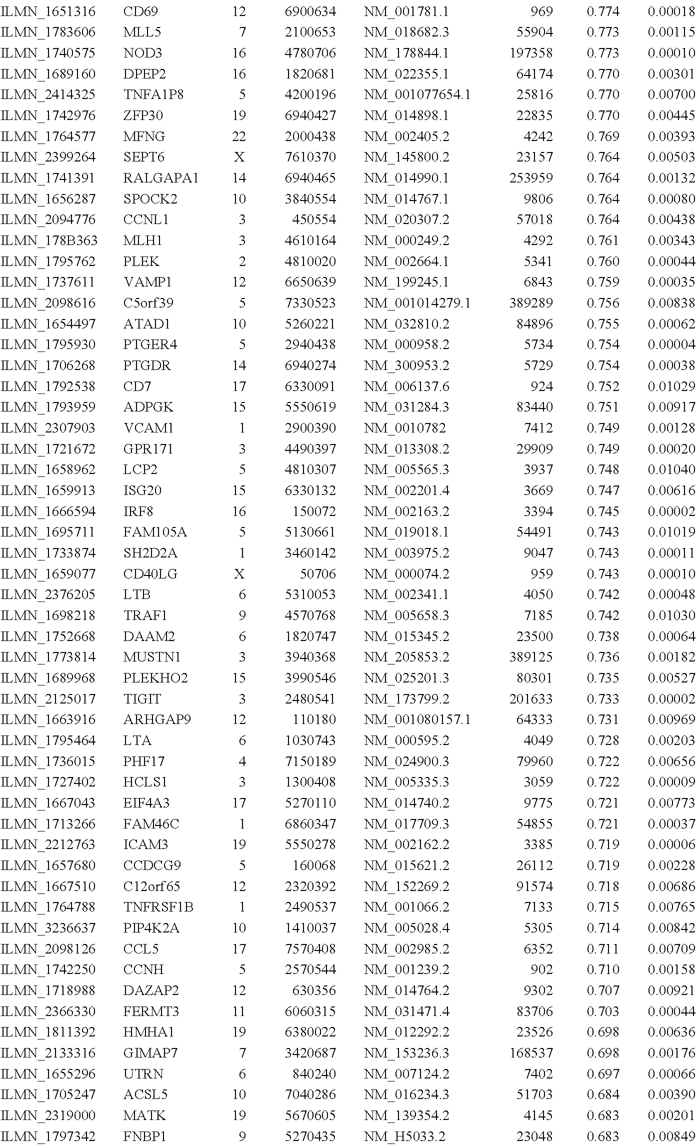

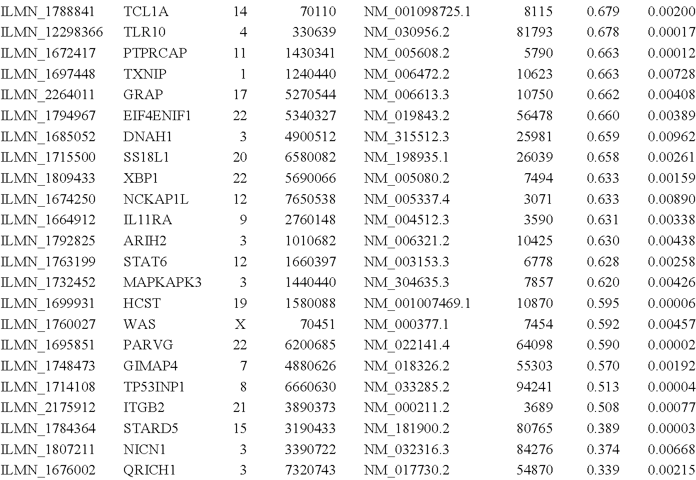

Regarding Table 5, adjusted HRs for genes associated with decreased RFS (top section) and increased RFS (bottom section) at p<0.01 were determined as described herein.

### Functional Analyses

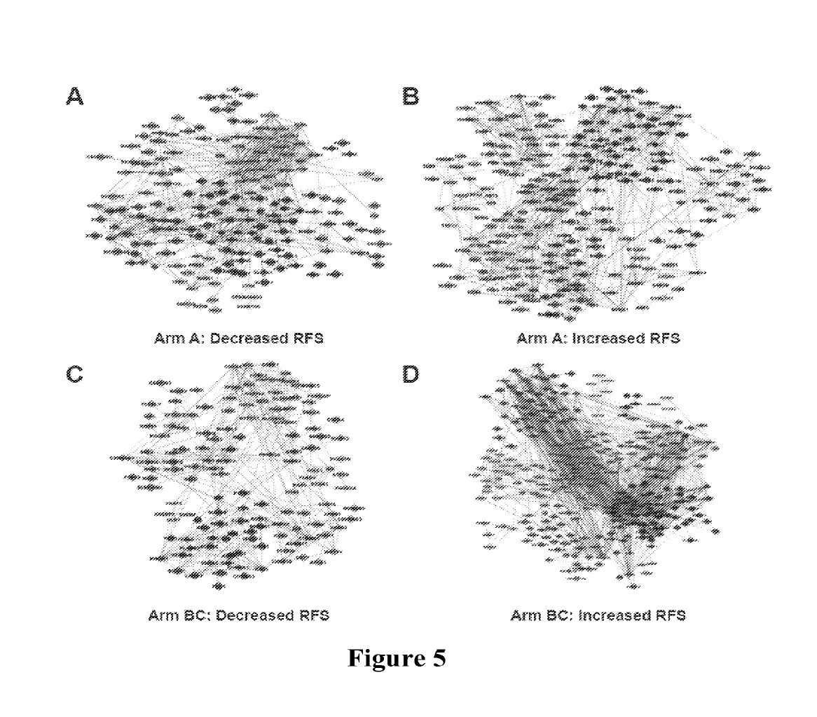

Cytoscape Functional Interactome tools were used to construct four interactome models using genes significantly associated with outcome (FIG. 5). Each interactome map contained 10-12 highly interconnected modules (color coded) that were connected to other modules within the networks. Pathway enrichment statistics were used to assess the biological significance of these four network models. The top-scoring pathways for each network are provided in Table 6. The most significant pathways associated with decreased RFS (HR>1.0) in Arm A were integrin signaling, co-regulation of androgen receptor activity, and vascular smooth muscle contraction (Table 6, panel A). Pathways associated with increased RFS (HR<1.0) in Arm A included formation and maturation of mRNA transcript, ribosome, neuroactive ligand-receptor interaction, homologous recombination, and innate immunity signaling (Table 6, panel B).

TABLE 6
Pathway enrichment statistics from Cytoscape networks. Significant
pathways were filtered for p < 0.001 and FDR < 0.1. Pathways were ranked on number
of genes from network in the individual pathways.

| | | | | | |
|---|---|---|---|---|---|
| | | Protein | | | |
| | | From | | | |
| Module | Geneset | Network | P-value | FDR | Nodes |

A. ArmA_Decreased RFS Pathways

| | | | | | |
|---|---|---|---|---|---|
| 1 | Integrin signaling pathway(P) | 9 | 0 | <1.00e−03 | COL18A1, COL4A1, COL13A1, JTGB3, |
| | | | | | LAMC3, COL6A3, COL1A2, |
| | | | | | COL6A1, COL10A1 |
| 0 | Coregulation of Androgen | 5 | 0 | <1.00e−03 | FHL2, LATS2, HIP1, KLK2, TGFB1I1 |
| | receptor activity(N) | | | | |
| 3 | Vascular smooth muscle | 3 | 0.0002 | 0.04 | PPP1R12B, MYL9, GUCY1B3 |
| | contraction(K) | | | | |

B. ArmA_Increased RFS Pathways

| | | | | | |
|---|---|---|---|---|---|
| 3 | Formation and Maturation of | 5 | 0 | <1.00e−03 | DHX9, NHP2L1, HNRNPR, HNRNPH2, |
| | mRNA Transcript(R) | | | | PHF5A |
| 4 | Ribosome(K) | 4 | 0 | <1.00e−03 | RPS4Y1, RPL23A, RPS5, RPS7 |
| 5 | Neuroactive ligand-receptor | 4 | 0.0003 | 0.1 | P2RY10, ADRB1, CYSLTR1, CHRM2 |
| | interaction(K) | | | | |
| 2 | Homologous recombination(K) | 3 | 0 | 0.0 | RAD51C, XRCC2, RAD51 |
| 8 | Innate Immunity Signaling(R) | 3 | 0 | 0.0 | TIRAP, ECSIT, TLR8 |

C. ArmBC_Decreased RFS Pathways

| | | | | | |
|---|---|---|---|---|---|
| 1 | Integrin signaling pathway(P) | 7 | 0 | <1.00e−03 | LIMS1, BCAR1, JTGA11, ELMO2, |
| | | | | | LAMB3, LAMC2, MAPK8 |
| 3 | Alzheimer disease-presenilin | 4 | 0 | 0.01 | JUP, ADAM17, WNT11, LDLRAD3 |
| | pathway(P) | | | | |
| 2 | M/G1 Transition(R) | 3 | 0.0001 | 0.01 | PSMD14, PSMB7, MCM8 |

D. ArmBC_Increased RFS Pathways

| | | | | | |
|---|---|---|---|---|---|
| 5 | Cytokine-cytokine receptor | 14 | 0 | <2.50e−04 | CXCL9, CCL19, CXCR3, CCL5, |
| | interaction(K) | | | | CXCL12, CCR7, CCR6, CXCR5, |
| | | | | | CXCR4, CXCL13, CCR4, CCL21, |
| | | | | | CCR10, CCR2 |
| 0 | TCR signaling in naive CD8+ T | 12 | 0 | <3.33e−04 | CD8A, CD3G, CD3D, CD3E, CD80, |
| | cells(N) | | | | LCKLCP2, CD247, IL2RG, PTPRC, |
| | | | | | IL2R8, FYN |
| 2 | IFN-gamma pathway(N) | 8 | 0 | <1.00e−03 | STAT8, TFF3, PRKCA, TGFBR2, |
| | | | | | PIM1, PRKCH, PRKCQ, JRF4 |
| 4 | TNF receptor signaling | 8 | 0 | <1.00e−03 | TRAF1, PRF1, MAPKAPK3, TNFRSF1B, |
| | pathway(N) | | | | CCM2, GZMB, BIRC3, MAP3K14 |
| 3 | Call surface interactions at the | 7 | 0 | <1.00e−03 | ITGAL, ITGB2, CD46, INPP5D, AMICA1, |
| | vascular wall(R) | | | | SELP, SELL |
| 5 | Class I PI3K signaling events(N) | 6 | 0 | <2.00e−04 | CD72, BTK, CD40LG, PLCG2, CD79B, |
| | | | | | CD79A |

Among the trastuzumab-treated patients (Arms B/C), integrin signaling, Alzheimer disease-presenilin pathway, and M/G1 cell cycle transition pathways were the most significant pathways linked to decreased RFS (HR>1.0) (Table 6, pane C). The most significant pathways associated with increased RFS (HR<1.0) after adjuvant trastuzumab (Table 6, panel D) included cytokine-cytokine receptor interaction, T-cell receptor signaling in CD8⁺ T-cells, INF-gamma pathway, TNF receptor signaling pathway, cell surface interaction at the vascular endothelium, and class 1 PI3K signaling events. The observation that 4/6 significant pathways are linked to immunological functions strongly suggests an association between immune response and increased RFS in trastuzumab-treated patients with HER2-positive breast tumors.

Gene Ontology Biological Process terms were defined for each gene with a significant HR (adjusted-model p<0.01). Fisher's exact test was used to identify 13 GO biological process descriptors that exhibited significantly different distribution in Arms A and B/C at p<0.01 (Table 7); the most significant was immune response (GO:0006955_BP). Ten of 13 biological processes were linked to immune functions, including T-cell and B-cell responses, chemokine signaling and chemotaxis, and inflammation. These results suggest that a major immunological component is predictive of RFS among trastuzumab-treated patients with early stage HER2-positive breast cancer.

87 immune function genes, defined by the 10 immune function GO terms that were enriched in Arms B/C (Table 7) and associated with increased RFS (HR<1.0) at adjusted-model p<0.01, were identified (Table 8). To find which of these probes were potentially predictive, probes among the 87 immune function genes that had a significant interaction term (p<0.05) were selected. This resulted in a list of 14 genes (Table 9).

TABLE 7
R
Analysis of biological process defined by gene ontology (GO) terms reveals enrichment of immune
function terms in Arms B/C. Thirteen GO biological process terms were enriched in Arms B/C,
relative to Arm A. Ten of these, labeled with “R,” were linked to various immune functions. GO terms
associated with signal transduction or response to drug are labeled with “G” and “B,” respectively.

| | | No. genes | | | | |
|---|---|---|---|---|---|---|
| | No. genes | from Arms | Total genes | Total genes | Fisher p | |
| GO | from Arm A | B/C | in Arm A | in Arms B/C | value | GO_Name |
| GO:0006955_BP | 7 | 42 | 334 | 299 | 1.40E−07 | immune response R |
| GO:0050776_BP | 1 | 20 | 82 | 63 | 6.43E−06 | regulation of immune response R |
| GO:0007166_BP | 3 | 26 | 229 | 206 | 8.47E−06 | cell surface receptor signaling pathway G |
| GO:0050852_BP | 2 | 16 | 77 | 63 | 0.0006793 | T cell receptor signaling pathway R |
| GO:0050853_BP | 0 | 10 | 30 | 20 | 0.000797 | B cell receptor signaling pathway R |
| GO:0007165_BP | 36 | 70 | 1294 | 1260 | 0.0009845 | signal transduction G |
| GO:0031295_BP | 2 | 15 | 63 | 50 | 0.001167 | T cell costimulation R |
| GO:0006935_BP | 1 | 13 | 112 | 100 | 0.001338 | chemotaxis R |
| GO:0006954_BP | 10 | 28 | 273 | 255 | 0.003764 | inflammatory response R |
| GO:0006968_BP | 1 | 11 | 54 | 44 | 0.003991 | cellular defense response R |
| GO:0042493_BP | 6 | 21 | 353 | 338 | 0.006068 | response to drug B |
| GO:0002407_BP | 0 | 7 | 13 | 6 | 0.005217 | dendritic cell chemotaxis R |
| GO:0070095_BP | 0 | 7 | 25 | 18 | 0.009625 | chemokine-mediated signaling pathway R |

TABLE 8
A cohort of 87 immune function genes are associated with RFS
in N9831. As listed in Table 7, 10 GO terms associated with
various immune functions were identified as enriched in a
comparison of Arm A versus Arms B/C. All genes with
significant HRs (p < 0.01) in either arm were then used to
generate a list of 87 immune function genes that are significantly
associated with RFS in either or both arms.
87 genes from top 10 GO biological
processes related to immune function.

| Symbol | Probe ID | HR_B/C | p-value |
|---|---|---|---|
| HLA-E | 1030747 | 0.445 | 0.00319 |
| ITGB2 | 3890373 | 0.508219 | 0.000768 |
| IGFBP4 | 7510414 | 0.557 | 0.00632 |
| WAS | 70451 | 0.592464 | 0.00457 |
| HCST | 1580686 | 0.594525 | 6.26E−05 |
| NCKAP1L | 7650538 | 0.632828 | 0.008901 |
| XBP1 | 5690066 | 0.632856 | 0.001587 |
| TLR10 | 380639 | 0.677583 | 0.000173 |
| CCL5 | 7570406 | 0.710536 | 0.007088 |
| TNFRSF1B | 2490537 | 0.714519 | 0.00765 |
| ICAM3 | 5550278 | 0.719198 | 6.05E−05 |
| KLRC1 | 3830575 | 0.725 | 0.00306 |
| LTA | 1030743 | 0.72809 | 0.00203 |
| IFNG | 360725 | 0.736 | 0.00524 |
| LTB | 5310053 | 0.742352 | 0.00048 |
| CD40LG | 50706 | 0.742507 | 9.78E−05 |
| IRF8 | 150072 | 0.744762 | 2.3E−05 |
| VCAM1 | 2900390 | 0.749079 | 0.00128 |
| PTGDR | 6940274 | 0.754088 | 0.000384 |
| PTGER4 | 2940438 | 0.754146 | 4.31E−05 |
| CD90 | 5390239 | 0.774744 | 0.000361 |
| CCR2 | 1660615 | 0.777609 | 0.00378 |
| SH2D1A | 5910465 | 0.77772 | 0.00064 |
| INPP5D | 3130669 | 0.780308 | 0.007807 |
| TLR9 | 1820440 | 0.780726 | 0.000269 |
| CD79A | 3940504 | 0.784439 | 0.005338 |
| CXCR3 | 4390202 | 0.784822 | 0.000113 |
| AOAH | 2940424 | 0.784923 | 0.0011 |
| BTK | 6380161 | 0.789328 | 0.008108 |
| P2RY14 | 1340364 | 0.789552 | 0.005788 |
| AMICA1 | 1580465 | 0.792681 | 4.39E−05 |
| CCR6 | 5898470 | 0.795241 | 0.000317 |
| LYZ | 1690056 | 0.797393 | 0.00244 |
| LY75 | 430215 | 0.80483 | 0.008759 |
| CXCR4 | 2600152 | 0.805237 | 0.009395 |
| ITGAL | 4180494 | 0.805723 | 0.006328 |
| CD3D | 1580411 | 0.806489 | 0.003733 |
| PTPRC | 6180288 | 0.809112 | 0.001383 |
| CD97 | 6960630 | 0.811109 | 0.002369 |
| IL2RG | 6450390 | 0.81394 | 0.001465 |
| KCNJ10 | 4480110 | 0.817543 | 0.008108 |
| LCK | 130161 | 0.819764 | 0.001104 |
| CD1E | 5490403 | 0.821425 | 0.000753 |
| FYN | 6290725 | 0.822806 | 0.00014 |
| CD160 | 2190019 | 0.823244 | 0.00652 |
| SPN | 10356 | 0.823887 | 0.004451 |
| PLCG2 | 2480424 | 0.826461 | 0.008404 |
| HLA-DPB1 | 1050360 | 0.826046 | 0.001187 |
| KLRC3 | 1070487 | 0.827017 | 0.00218 |
| NCR1 | 5080288 | 0.827988 | 0.003448 |
| IDO1 | 5570711 | 0.830082 | 0.000743 |
| CD3E | 1780600 | 0.83051 | 0.001482 |
| PTPN22 | 6580044 | 0.834469 | 0.007923 |
| HLA-DOB | 3450338 | 0.835456 | 0.003544 |
| CCR4 | 7570154 | 0.837346 | 0.000252 |
| CCR10 | 2680753 | 0.837619 | 0.002636 |
| CD247 | 3890689 | 0.839228 | 0.003275 |
| CXCL13 | 1110564 | 0.8423 | 0.000391 |
| SLA2 | 4230671 | 0.845397 | 0.001099 |
| GZMA | 3420612 | 0.845874 | 0.001019 |
| C3 | 4880494 | 0.84898 | 0.001833 |
| ENPP2 | 840678 | 0.849319 | 0.009852 |
| TCF7 | 240494 | 0.851001 | 0.006267 |
| CD3G | 109047 | 0.851934 | 0.009728 |
| CXCL12 | 3870253 | 0.852965 | 0.001036 |
| CD96 | 2710754 | 0.854191 | 0.00021 |
| LAX1 | 580411 | 0.854971 | 0.00759 |
| CD79B | 4280725 | 0.856939 | 0.002844 |
| CD274 | 4900239 | 0.856941 | 0.008923 |
| CD38 | 2760500 | 0.857548 | 0.001349 |
| PRF1 | 4670193 | 0.860086 | 0.007283 |
| CTLA4 | 6400333 | 0.861406 | 0.004521 |
| AFAP1L2 | 4590133 | 0.861505 | 0.006674 |
| PRKCQ | 4640576 | 0.865456 | 0.009007 |
| APOL3 | 460327 | 0.865772 | 0.001638 |
| CCR7 | 5390246 | 0.866622 | 0.000507 |
| ICOS | 2070037 | 0.866813 | 0.006614 |
| SELL | 6940358 | 0.869716 | 0.000575 |
| IL7R | 3830349 | 0.870524 | 0.002977 |
| KLRG1 | 4880193 | 0.876006 | 0.008335 |
| IGLL1 | 4730747 | 0.876643 | 0.008782 |
| ITK | 7550632 | 0.877154 | 0.002111 |
| CD8A | 3170128 | 0.878357 | 0.003781 |
| TNFRSF13C | 2340753 | 0.882506 | 0.004797 |
| CCL21 | 1340626 | 0.899178 | 0.001933 |
| SELP | 4810468 | 0.912319 | 0.009693 |
| CCL19 | 7100646 | 0.914852 | 0.002751 |

TABLE 9
Interaction p-values. The table displays the hazard ratios (HRs) for the probe expression effect (HR.exprs), treatment arm effect
(HR.rand.arm), and the interaction of probe and treatment arm (HR interaction exprs:arm) in a multivariable Cox model that also
contained prognostic variables (nodal status, tumor size, hormone receptor status, age, and tumor grade) as adjusting variables.
The prognostic adjusting variables are not shown in the table. It also includes the p-values for the probe expression, treatment
arm, and the probe-treatment arm interaction variables: p.exprs, p.rand.arm, and p interaction exprs:arm, respectively.

| | | | | | Adjusted CoxPH Model Results ----->>>> | | | | | |
|---|---|---|---|---|---|---|---|---|---|---|
| | | | | | | | | | HR | p |
| | | | | ENTREZ_{—} | | | | | interaction | interaction |
| feature.id | SYMBOL | CHR | REFSEQ_ID | GENE_ID | HR.exprs | p.exprs | HR.rand.arm | p.rand.arm | exprs: arm | exprs: arm |
| ILMN_1730995 | AFAP1L2 | 10 | NM_001001936.1 | 84632 | 1.159 | 0.06452 | 12.308 | 0.01438 | 0.751 | 0.00338 |
| ILMN_2298366 | TLR10 | 4 | NM_030956.2 | 81793 | 1.020 | 0.80077 | 10.079 | 0.01511 | 0.669 | 0.00339 |
| ILMN_2249920 | FYN | 6 | NM_002037.3 | 2534 | 1.043 | 0.50014 | 5.423 | 0.02193 | 0.784 | 0.00344 |
| ILMN_1659077 | CD40LG | X | NM_000074.2 | 959 | 1.012 | 0.87277 | 6.022 | 0.02537 | 0.729 | 0.00480 |
| ILMN_1666594 | IRF8 | 16 | NM_002163.2 | 3394 | 0.978 | 0.78847 | 12.057 | 0.02353 | 0.742 | 0.00763 |
| ILMN_1803825 | CXCL12 | 10 | NM_000609.4 | 6387 | 1.045 | 0.46139 | 3.804 | 0.06953 | 0.821 | 0.01369 |
| ILMN_2066143 | CCR4 | 3 | NM_005508.4 | 1233 | 1.034 | 0.56977 | 3.338 | 0.07787 | 0.824 | 0.01387 |
| ILMN_1677505 | CCL21 | 9 | NM_002989.2 | 6366 | 1.037 | 0.40618 | 2.890 | 0.09652 | 0.873 | 0.01571 |
| ILMN_1665865 | IGFBP4 | 17 | NM_001552.2 | 3487 | 1.475 | 0.15634 | 95278.384 | 0.02531 | 0.447 | 0.02003 |
| ILMN_1778723 | AMICA1 | 11 | NM_153206.1 | 120425 | 0.967 | 0.58797 | 4.004 | 0.07809 | 0.818 | 0.02053 |
| ILMN_1706268 | PTGDR | 14 | NM_000953.2 | 5723 | 1.010 | 0.89629 | 3.993 | 0.08824 | 0.775 | 0.02306 |
| ILMN_1795930 | PTGER4 | 5 | NM_009958.2 | 5734 | 1.028 | 0.79413 | 13.251 | 0.05794 | 0.753 | 0.02623 |
| ILMN_2335754 | CD1E | 1 | NM_001042586.1 | 913 | 1.005 | 0.94079 | 3.119 | 0.13919 | 0.823 | 0.03688 |
| ILMN_1700428 | HLA-DOB | 6 | NM_002120.3 | 3112 | 1.022 | 9.75665 | 4.117 | 0.13916 | 0.829 | 0.04921 |

### Voting Scheme Parameters

The response surface analysis resulted in two unique sets of q/m values. The first set q=40 and m=9 (q40m9) occurred 235 times (47%) and identified 226 (52.2%) enriched patients in Arm A and 441 (51.9%) enriched patients in Arms B/C. The second set q=58 and m=8 (q58m8) occurred 265 times (53%) and identified 139 (32.1%) enriched patients in Arm A and 310 (36.5%) enriched patients in Arms B/C. Since both sets of optimum q/m values occurred about evenly, q/m pair q40m9 was selected as the optimum.

### Final Signature Analysis

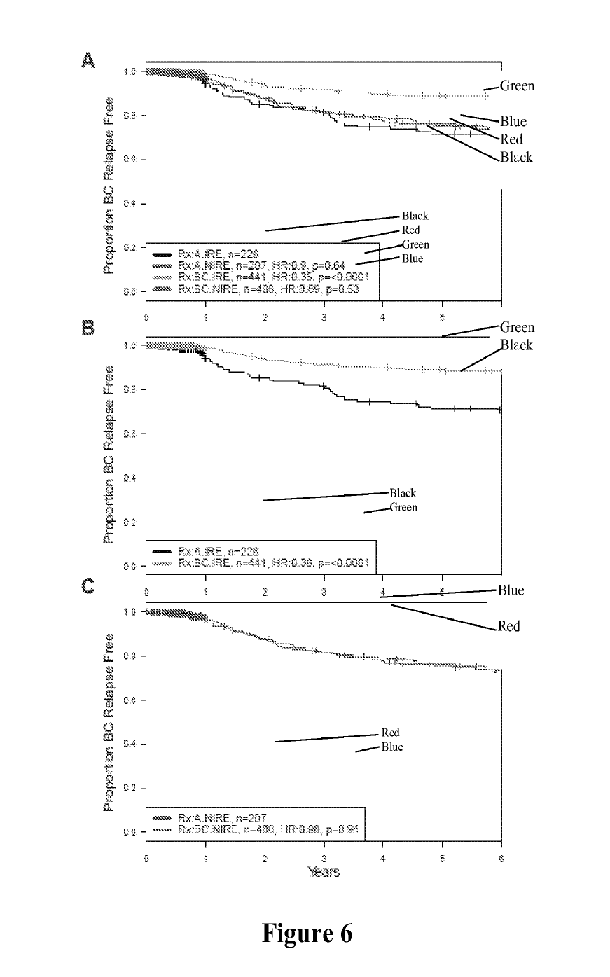

Based on the optimum set of q/m values, a tumor was designated as immune-enriched if any 9 (m) or more of the 14 immune function genes were expressed at or above the 0.40 quantile (q) expression value for one or more probes. This signature was used to “bin” tumors in Arm A and Arms B/C into immune response enriched (IRE) and non-immune response enriched (NIRE) groups. The difference in RFS between the IRE and NIRE tumors in Arm A was not statistically significant (HR=0.90, p=0.64, black and red labeled curves, FIG. 6A). Patients with IRE tumors exhibited significantly increased RFS after adjuvant trastuzumab (green labeled curve), compared to IRE patients who did not receive trastuzumab (black labeled curve; HR=0.35, p<0.0001). Furthermore, the RFS of trastuzumab-treated patients whose tumors were NIRE (blue labeled curve) was not significantly different from RFS of IRE patients who received chemotherapy alone (HR=0.89, p=0.53). A multivariable Cox model was evaluated that included the prognostic factors as adjusting variables, immune-enrichment status, treatment group, and an immune-enrichment status and treatment group interaction group term. In this model, the interaction term value was significant (p<0.0001). FIGS. 6B and 6C show the effect of the interaction on trastuzumab response. There is a difference in RFS for patients with IRE tumors treated with trastuzumab compared to those who received chemotherapy alone (FIG. 6B; HR=0.36, p<0.0001). There is no difference in RFS for patients with NIRE tumors treated with trastuzumab and those who received chemotherapy alone (FIG. 6C; HR=0.98; p=0.91).

### Cross-Validation Results

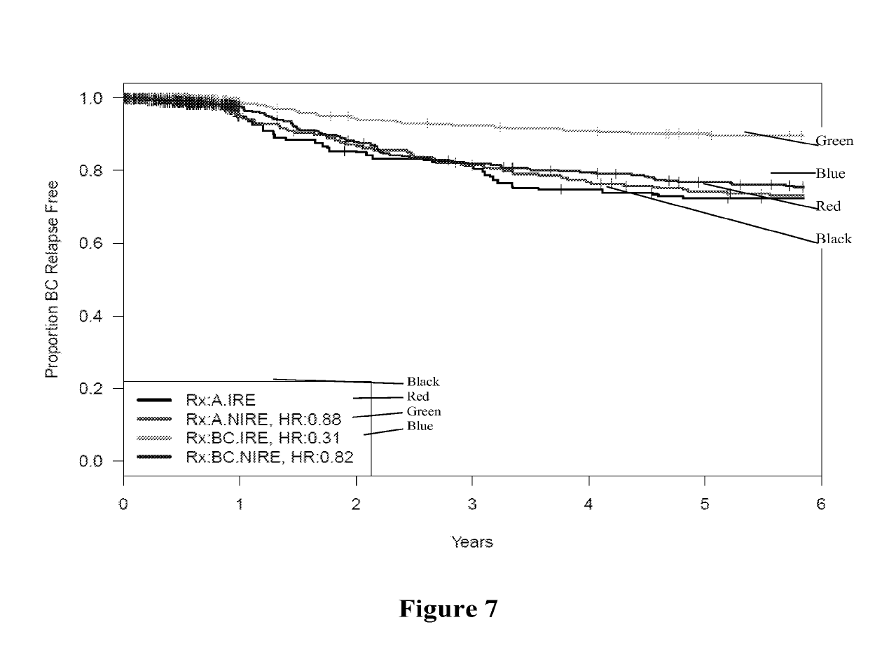

To validate the signature, a five-fold cross validation was performed. The immune enrichment status of the tumors in the “left out” groups for each iteration were combined, so that all samples within the study were assigned as enriched or non-enriched. FIG. 7 shows the RFS curves for each enrichment status and treatment group combination obtained from cross-validation. There is no difference in RFS between Arm A and Arms B/C for NIRE tumors (HR=0.93), but there is a difference in RFS between Arm A and Arms B/C for IRE tumors (HR=0.32). The p-value for the enrichment status-treatment group interaction was less than 0.0001 in the multivariable Cox model that adjusted for known prognostic factors.

These results demonstrate that when nine or more of the fourteen immune function genes listed in Table 9 are at or above 0.40 quantile for the population for a particular patient, then that patient has an increased likelihood of remission free survival following treatment with adjuvant trastuzumab.

### Example 2

### Treating HER2-Positive Breast Cancer with Trastuzumab

A patient with HER2-positive breast cancer is identified as having an increased level of expression of nine or more of the fourteen genes listed in Table 9 and is administered a taxane agent (e.g., paclitaxel) and trastuzumab. The taxane agent is administered at a dose that is between 80 and 100 mg/m² per week. Trastuzumab is administered at a dose that is 2 mg/kg every week or 6 mg/kg every 3 weeks (after loading doses).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-12. (canceled)

13. A method for treating breast cancer, wherein said method comprises:
(a) detecting the presence of an elevated level of expression for at least nine of the nucleic acids listed in Table 9 in breast cancer cells from a mammal, and
(b) administering a taxane compound and trastuzumab to said mammal under conditions wherein the number of breast cancer cells within said mammal is reduced.

14. The method of claim 13, wherein said mammal is a human.

15. The method of claim 13, wherein said elevated levels are determined using a cDNA-mediated annealing, selection, extension, and ligation (DASL) assay.

16. The method of claim 13, wherein said breast cancer is an HER2-positive breast cancer.

17. The method of claim 13, wherein said taxane compound is paclitaxel.
